(19) United States
(12) Patent Application Publication
(10) Pub. No.: US 2021/0395722 A1
(43) Pub. Date: Dec. 23, 2021

(54) TTF Generated Proliferation of Cytotoxic T Cells to Create a Specific Pro-Inflammatory Response

(71) Applicant: NOVOCURE GMBH, Root D4 (CH)

(72) Inventors: Tali Voloshin-Sela, Kibbutz Gvat (IL); Lilach Avigdor, Haifa (IL)

(21) Appl. No.: 17/351,343

(22) Filed: Jun. 18, 2021

Related U.S. Application Data
(60) Provisional application No. 63/041,211, filed on Jun. 19, 2020.

(51) Int. Cl. C12N 13/00; C12N 5/0783; A61K 35/17; A61N 1/32

(52) U.S. Cl. CPC C12N 13/00; C12N 5/0638; C12N 2529/00; A61N 1/326; A61K 35/17

(57) ABSTRACT

Disclosed are methods of increasing proliferation of CD8+ T cells comprising exposing a target site to an alternating electric field for a period of time, the alternating electric field having a frequency and field strength, wherein the frequency and field strength of the alternating electric field increases proliferation of CD8+ T cells at the target site. Disclosed are methods of generating a pro-inflammatory response in a target site comprising exposing the target site to an alternating electric field for a period of time, the alternating electric field having a frequency and field strength, wherein the frequency and field strength of the alternating electric field generates a pro-inflammatory response at the target site. Disclosed are methods of increasing proliferation of CD8+ T cells comprising exposing CD8+ T cells to an alternating electric field for a period of time, the alternating electric field having a frequency and field strength, wherein the frequency and field strength of the alternating electric field increases proliferation of CD8+ T cells. Disclosed are methods of treating a subject in need of a CD8+ T cell response comprising applying an alternating electric field to a target site of the subject for a period of time, the alternating electric field having a frequency and field strength, wherein the target site comprises one or more CD8+ T cells.

### CROSS-REFERENCE TO RELATED APPLICATIONS

This application claims priority to U.S. Provisional Patent Application No. 63/041,211, filed on Jun. 19, 2020, which is incorporated by reference herein in its entirety.

### BACKGROUND

Tumor Treating Fields (TTFields) represent a locoregional tumor treatment that uses alternating electric fields tuned to target proliferating cells. The fields exert an electromotive force on highly polar proteins cardinal to the mitotic phase of cell division, thereby disrupting mitotic spindle formation and charged organelle translocation during mitosis. These physical interactions result in inhibiting proliferation and in inducing cancer cell death.

Optune® is a medical TTFields delivery device that has demonstrated anti-mitotic effects in preclinical and clinical research on several solid tumors. Optune has been FDA-approved for treatment of recurrent and newly diagnosed glioblastoma (GBM), where, when delivered concurrently with standard chemoradiation therapy, it was shown to significantly prolong progression-free and overall survival while maintaining good quality of life. It was approved for treatment of malignant pleural mesothelioma in 2019, and its efficacy is currently being tested in phase 3 clinical trials for non-small cell lung cancer (NCT02973789), ovarian (NCT03606590), pancreatic (NCT03377491), liver (NCT03606590) and gastric cancers (NCT04281576). Notwithstanding, even following TTFields' combination therapy, the median survival rate of newly diagnosed GBM patients is 20.8 months highlighting the need for more effective multimodal treatments.

Cancer immunotherapy has emerged as a central arm in battling malignancies, and has been the focus of a great number of recent studies. Responses to immunotherapy positively correlate with tumor neoantigen load, PDL1 expression and the abundance of tumor-infiltrating T lymphocytes (TILs) found in immunologically “hot” tumors. Unfortunately, with tumors such as gliomas, which have low neoantigen load and are immunologically “cold”, the majority of cancer patients fail to respond to immunotherapy.

A common approach to increasing responsiveness to immunotherapy is to combine treatments which transform “colder” tumors into “hotter” ones, which are more amenable to immunotherapy. An example of such a combination is localized irradiation and the use of checkpoint inhibitors (CPIs). Irradiation therapy has been shown to generate immunogenic cell death (ICD), a process in which dying cells present danger-associated molecular patterns. These signals enhance antigen presentation by dendritic cells and promote priming, activation and trafficking of T cells to the tumor. Addition of CPIs may facilitate the priming or the ongoing activation of the induced T cells. TTFields locoregional treatment may induce a similar in-situ vaccine effect on tumors.

### BRIEF SUMMARY

The immune system may play an important role in mediating the clinical effects of TTFields, plausibly by their induction of ICD in dying cells and STING pathway activation. TTFields is a locoregional treatment and thus expected to cause minimal or no systemic immunosuppression. TTFields was shown to reduce distant metastatic spread to the lungs in a rabbit renal cancer model, and to enhancing peri- and intratumoral infiltration of CD4+, CD8+ T cells. It was shown to enhance antitumor efficacy when combined with anti-PD1 in mice models. Interestingly, in a retrospective study in GBM patients, the overall survival benefit of TTFields was found to correlate with higher blood T-cell counts and with low (<4.1 mg) or no administration of immunosuppressive dexamethasone.

Trials combining TTFields with checkpoint inhibitors (NCT02973789, NCT03405792, NCT03903640, NCT03430791) or other immunotherapeutics (NCT03223103) are underway. However, it is yet unknown what is the direct effect of TTFields on T cells as the main drivers of anti-tumoral responses within the TTFields-treated field. Understanding whether T cells can generate an immune response under the effect of TTFields is therefore of cardinal mechanistic importance to current and future combination trials.

Disclosed are methods of increasing proliferation of CD8+ T cells comprising exposing a target site to an alternating electric field for a period of time, the alternating electric field having a frequency and field strength, wherein the frequency and field strength of the alternating electric field increases proliferation of CD8+ T cells at the target site.

Disclosed are methods of generating a pro-inflammatory response in a target site comprising exposing the target site to an alternating electric field for a period of time, the alternating electric field having a frequency and field strength, wherein the frequency and field strength of the alternating electric field generates a pro-inflammatory response at the target site.

Disclosed are methods of increasing proliferation of CD8+ T cells comprising exposing CD8+ T cells to an alternating electric field for a period of time, the alternating electric field having a frequency and field strength, wherein the frequency and field strength of the alternating electric field increases proliferation of CD8+ T cells.

Disclosed are methods of treating a subject in need of a CD8+ T cell response comprising applying an alternating electric field to a target site of the subject for a period of time, the alternating electric field having a frequency and field strength, wherein the target site comprises one or more CD8+ T cells.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A and B show effects of TTFields on the viability of peripheral blood T cells. CFSE-stained healthy donor PBMC were treated with PHA or left untreated and incubated under TTFields or standard conditions for 3.5 days. A. Flow cytometry gating strategy for unstimulated (left) and PHA-stimulated (right) cells. Activation with PHA markedly changed cell sizes and maker expression levels and variance within populations requiring separate gating. Singlet discrimination (x2)→removal of events with non-specific fluorescence→B cell removal→monocyte removal→gating of T cells→classification as CD4⁺8⁻ Th or CD8⁺4⁻ CTL→categorization of four groups according to viability (ViViD) and proliferation (CFSE) status. B. Relative T-cell numbers and viability rates in stimulated and unstimulated samples, according to proliferative status. In each repeat, data were normalized by setting the value of live, non-proliferating cells in control samples to 1. Stacked bars show normalized numbers of live cells. Viability rates (#live/#total) appear under each bar. Graphs show the mean of 8 independent repeats using 4 different PBMC samples.

FIGS. 2A-2D show effects of TTFields on the activation of blood-borne T cells. CFSE-stained PBMC were treated with PHA or left untreated, and incubated under TTFields or standard conditions for 3.5 days. A. Gating strategy: Singlet discrimination (x2)→gating of viable (ViViD^{dim}) T cells→classification as CD4⁺8⁻ (Th), CD8⁺4⁻ (CTL) or CD4-CD8- T cells (consisting of ˜90% Tγδ in healthy blood). Each subset was evaluated for surface expression of PD1 and CD107a for proliferation (CFSE) and IFNγ production. B-C: Net activation per each function was calculated by subtracting % positive cells in the unstimulated sample from that of the matched stimulated sample. Graphs show the mean of 9-11 independent repeats using samples from 6 different donors. B. Summary of functional responses grouped per single function. C. Summary of polyfunctional analysis. Cells were divided into 15 mutually exclusive functional groups according to each individual cell's expression of one or more functions. The net percent of polyfunctional-positive cells is presented. D. Analysis of T cell viability in activated cells. Total number of live T cells in all samples (I) and in PHA-stimulated samples only (II). A polyfunctional analysis was used to count ‘activated non-proliferating cells’ which were negative for proliferation but positive for at least one other functional parameter. Graphs III, IV: cell numbers of activated, non-proliferating CD4⁺ and CD8⁺ T cells, respectively.

FIGS. 3A-3C show an effect of TTFields on activation of human GBM TILs. Fresh GBM samples enzymatically dissociated to viable single cells were stained with CFSE, stimulated by PHA or left untreated, and then incubated under TTFields or standard conditions for 3.5 days. Cells were harvested and stained as in FIG. 2. Donor PBMC samples (not shown) were treated identically and used as guides for gating of flow cytometric data (FIG. 7A). Net activation was calculated by subtracting % positive cells in the unstimulated sample from that of the matched stimulated sample. The graphs show means and standard error of three independent repeats A. Summary of specific functional responses in CD4⁺ and CD8⁺ T cells. B. Summary of polyfunctional analysis. Cells were divided into 15 mutually exclusive and distinct functional groups according to each individual cell's expression of one or more functions. The net percent of polyfunctional-positive cells is presented C. Relative rates and polyfunctionality of PD1⁺ TILs (TASTs) in the non-stimulated samples. The PD1⁺ T cells were divided into eight distinct polyfunctional groups according to their expression of one or more functions in individual cells.

FIGS. 4A and 4B show an effect of TTFields on direct cytotoxicity of CAR-T cells. Effector human anti-HER2 CAR T cells and target CAG multiple myeloma cells (CD138⁺), either expressing (CAG-Her2) or not expressing (CAG) HER2, were co-cultured under TTFields or control conditions for 8 hours. Target cells were also cultured alone to determine background cell death. A. Gating strategy: singlet discrimination→gating of target cells by FSC/SSC→gating of target cells by CD138⁺CD3⁻→gating out GFP⁺ expressing CAR effectors→cellular viability by propidium iodide (PI). B. Specific killing curves: the dead cell fraction in ‘target only’ samples was subtracted from the concordant samples (lighter vs. darker lines, respectively). Net specific killing (black line) was calculated by subtracting the fraction of killed cells in the CAG samples from that in the CAG-HER2 samples. Representative results (CAR transduction efficiency ˜35%) from one assay are shown. Similar results were observed in three repeats with varying killing rates due to differences between individual PBMC donors and individual CAR transduction efficiencies.

FIGS. 5A-5C show T cell infiltration rates and immunity-related gene expression in GBM tumors treated with TTFields therapy. GBM tumor samples were obtained from four patients before and after treatment with TTFields combined with standard chemoradiation. Tissues were stained for nuclear visualization and stained by immunohistochemistry (IHC) for CD3, CD4 and CD8. A. Cells were counted in 4-6 representative fields in each slide and cell count/mm² was calculated. The charts show calculated mean densities of CD3+, CD4+ and CD8+ cells for each patient individually, before and after TTFields therapy. B. Representative IHC images of tissue section from patient 4*. Positive cells are stained brown. C. GBM tumor samples were obtained before and after a treatment according to a standard chemoradiation protocol (six patients) or a protocol combining TTFields with standard chemoradiation (six patients). Gene expression analysis was performed by RNA-seq. The negative binomial generalized linear model was used to analyze expression following treatment and the differential effects of control and TTFields treatments. A list of 712 immune activity-related genes was evaluated. A significant effect of TTFields on expression was defined as an average fold change of either >2 or <0.5, with a p value <0.1. Table 5C lists the genes exhibiting significantly altered expression. Each gene was designated as having primarily pro-/anti-/mixed tumoral activity based on a review of the relevant literature.

FIGS. 6A and 6B show a media conditioning assay for validation of inovitro® procedure adjusted for non-adherent cultures. The field intensity in the Inovitro cultures is derived from the current that the Inovitro system automatically determines in order to maintain the TTFields-heated culture dishes at 37° C. The incubator temperature was calibrated to 28.3° C., yielding currents in the range of 75-95 mA and the desired field intensity specified in the Methods section of the main text. The difference in temperatures between the culture media and the incubator produced accelerated evaporation. As the evaporated fraction is essentially pure water, replenishment of evaporated volume by daily addition of double-distilled water (DDW) was chosen. To evaluate the effects of media evaporation and the method selected to solve this issue, X-vivo 15 defined cell culture media were preconditioned by incubation of 2 ml aliquots in inovitro culture dishes for 3 days. Dishes were incubated at 37° C. either in a standard incubator or in the inovitro TTFields generator (at the above-specified parameters), or in an inovitro generator and with daily replenishment of the volume of media that had evaporated by sterilized double-distilled water (DDW). The pre-conditioned media were separately collected, along with fresh medium, and were used for culturing. PBMC were stained with CFSE, either stimulated by PHA or left unstimulated, and cultured for 3 days in standard culture plates with the different media collected. A. Light-field microscopy images from individual wells (x40) showing that TTFields conditioned media that was not unreplenished with DDW did not support T cell proliferation and cell clumping, while all other conditions did, including the DDW-replenished TTFields-conditioned media. B. Flow cytometric analysis of PBMC activation in preconditioned media and fresh media. The PHA-stimulated cells were collected and stained for viability (ViViD), T cell markers (CD3,CD4,CD8) and a selection of T cell activation markers (IL-2, PD-1, TNFα). CD4+ and CD8+ T cells were isolated in serial gating similarly to FIG. 2 and evaluated for activation marker expression. Results show that all media except the unreplenished TTFields-conditioned media generated similar activation responses. To control for DDW replenishment in the specified assay and in all reported assays, both the TTFields and the standard cultured samples seeded in inovitro dishes were replenished daily with DDW according to their respective evaporation volumes. Evaporation volumes were evaluated separately and periodically (every 4 months) by direct measurement of multiple dishes and deriving an average evaporation rate from either control or TTFields-treated dishes.

FIGS. 7A and 7B show effects of TTFields on activation of GBM TILs. A. Representative gating strategy for PHA-stimulated and unstimulated GBM TIL culture. PB samples were used for gate placement (not shown). Gating strategy consisted of singlet discrimination (x2) gating of live T cells as CD3⁺ViViD^{low}CD14⁻CD19⁻→division into CD4⁺8⁻ Th and CD8⁺4⁻ CTL. Each T cell subset was then evaluated for surface expression of PD-1 and CD107a, for CFSE dilution and for intracellular expression of IFNγ. Gating of Tγδ cells using CD3⁺CD4⁻CD8⁻ identified a lower percentage of Tγδ cells than in PB and thus was not used. B. Analysis of T cell viability in stimulated cells. (I) The fraction of live T cells in each PHA-stimulated TIL sample. Data from 3 repeats was normalized by setting the value of the control+PHA sample to 1. Polyfunctional analysis was used to enumerate the cells that were negative for proliferation but positive for at least one other activation parameter. Graphs represent the relative fractions of these cells within the CD4⁺ (II) and the CD8⁺ (III) T cell subsets. Results show no significant decline in the number of activated T cells which did not attempt to proliferate. The significant reduction noted in (I) thus consists mostly of the activated T cells which attempted to proliferate.

FIG. 8 shows an effect of TTFields on viability of effector cells used in cytotoxicity assay. Anti-Her2 CAR T-cells (GFP⁺) which served as effectors were evaluated for viability following 8 hours of culture under control or TTFields conditions. Cultures containing only CAR T cells were collected and stained by PI to detect cell death and for CD3 and CD138 (a CAG target marker, to also serve as a gating guide for main sample). Representative gating strategy consisted of singlet discrimination→isolation of effector cells by FSC/SSC→gating for CD138⁻CD3⁺ CAR effectors→gating for viability. Results show that TTFields have no effect on the viability of effector cells during assay duration. This validates that the noted net cytotoxicity was not influenced by an indirect effect of TTFields on the viability of the CAR T cells during the assay.

FIG. 9 shows a gene expression analysis of CD3, CD4 and CD8 in GBM tumors following TTFields therapy. Samples of GBM resections were taken from before and after a treatment course by standard chemoradiation (i.e. Stupp protocol, control) versus approved TTFields protocol (Optune+chemoradiation). Total RNA was purified and sequenced by NGS. RNAseq quality control check was performed using FastQC v0.11.5 (1). Reads were trimmed using Trimmomatic v0.36 (for removing sequencing adapters) (2). Reads were mapped to Homo sapiens genome (Grch38) using STAR read aligner v2.4.2a (default parameters) (3). Counting proceeded over genes annotated in Ensembl release 83, using featureCounts (4). Read counting, normalization and conversion to RPKM (5) were performed using edgeR v3.4.1 (6). Statistic analysis was carried out using the negative binomial generalized linear model. Charts show the difference in RPKM between post- and per-treatment expression for each patient by group (12 patients total). Group averages are shown as a black line with standard error bars.

FIGS. 10A and 10B show an example of the total numbers of viable CD4+ (A) and CD8+ (B) T cells under variable TTFields frequencies. Equal numbers of healthy donor PBMC were cultured for 3 days under TTFields or standard culture conditions, with or without Phytohemaglutinin (PHA) as a mitogen/superantigen. The cells were then collected and adjusted to 350 ul, then read for 2.5 minutes of FACS CANTO-II drawing equal volumes per minute/sample. Statistics—each sample was compared to the control standardly—cultured matched sample using t-test *P<0.005. Repeated 3 times with similar results; 100 and 150 KHz—2 repeats.

FIGS. 11A and 11B show changes in the fractions of functional proliferating or non-proliferating CD4 (A) or CD8+ (B) T cells. Equal numbers of healthy donor CFSE-stained PBMC were cultured for 3 days under TTFields or standard culture conditions, with or without Phytohaemagglutinin (PHA) as a mitogen/superantigen. The cells were collected and flow cytometrically evaluated for four concurrent functions: proliferation (CFSE dilution), cytotoxic degranulation (CD107 surface expression), IFNg secretion and PD1 upregulation (activation/exhaustion). Net percent positive cells was calculated by deducting the fraction of cells in the unstimulated sample from the matching group in the PHA-stimulated sample. Statistics: t-test *P<0.05 to matched standardly cultured control (frequency 0 kHz). Repeated 3 times with similar results; 100 and 150 KHz—2 repeats. Similar dataset as in FIG. 10.

FIG. 12 is a table showing a summary of patient data from FIGS. 5 and 9.

FIG. 13 is a table showing literature review of genes with significantly altered expression following TTFields treatment of GBM tumors.

FIGS. 14A-C show induction of anti-tumor immunity in GBM by TTFields requires STING and AIM2. Combo box and whisker and dot plots showing immunophenotyping for total DCs and fully activated (CD44+, CD62L−) CD4+ and CD8+ T cells in PBMCs of surviving Sc-TTF-immunized animals at 1 (a) and 2 (b) weeks post re-challenge with KR158-luc as compared to a new naïve cohort implanted with the same KR158-luc cells, and for central memory (CM) (CD44+, CD62L+) CD4+ and CD8+ T cells and their activated (effector) counterparts in dcLNs (c) in long-term surviving Sc-TTF-immunized animals at 20 weeks after re-challenge as compared to age-matched, sex-matched naïve mice implanted with the same KR158-luc cells for 2 weeks. All comparisons for the immunophenotyping were performed using one-way ANOVA for post primary immunization samples and Student's t-test with a 2-tailed distribution for the re-challenge samples; *, p<0.05.; **, p<0.01; ***, p<0.001.

FIG. 15 shows TTFields treatment correlates with activation of the immune system in GBM patients via a T1IRG-based trajectory. a) A diagram detailing adjuvant TTFields treatment in patients with newly diagnosed GBM. PBMCs were obtained immediately before and about 4 weeks after initiation of TTFields. Twelve patients were enrolled and their PBMCs were divided into 2 analytical groups: scRNA-seq and bulk RNA-seq of isolated T cells.

b) Colored cell cluster map at Resolution 1 using the graph-based cell clustering technique UMAP resolving 38 major immune cell types and subtypes in the scRNA-seq dataset of PBMCs in 12 GBM patients. c) A heatmap of expression levels of the indicated gene set implicated in various T cell fates and functions providing the basis for annotations of the indicated major T cell clusters. d) An overlay of pre-TTFields (pre-TTF—green) and post-TTF (orange) UMAP plots showing post-TTF changes in both proportions and expression (purple broken line) and expression only without proportional change (blue broken line) of the indicated key clusters. e) A heatmap of mean expression levels of the T1 IRG pathway GO:0034340 at the single cell level in pre-TTF and post-TTF PBMCs. f-l) Combo box and whisker and paired dot plots showing the proportions of the indicated clusters as percent of total PBMCs in pre-TTF and post-TTF PBMCs. Analysis was performed using the Wilcoxon test. m and o) Heatmaps of gene expression showing log FC of post-TTF expression of all-genes compared to pre-TTF expression of all genes in pDCs (m) and cDCs (o) in patients with detectable pre- and post TTF counts in the respective cell types. n and p) Gene set enrichment analysis (GSEA) of the indicated GO pathways in pDCs (n) and cDCs (p) comparing between pre and post TTFields treatment of the sample patients in m and o. NES: normalized enrichment score.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “an alternating electric field” includes a plurality of such alternating electric field, reference to “the target site” is a reference to one or more target sites and equivalents thereof known to those skilled in the art, and so forth.

As used herein, a “target site” is a specific site or location within or present on a subject or patient. For example, a “target site” can refer to, but is not limited to a cell, population of cells, organ, tissue. In some aspects, a cell or population of cells can be one or more T cells. In some aspects, a “target site” can be a site of inflammation or a site comprising a draining lymph node.

As used herein, an “alternating electric field” or “alternating electric fields” refers to a very-low-intensity, directional, intermediate-frequency alternating electrical fields delivered to a subject, a sample obtained from a subject or to a specific location within a subject or patient (e.g. a target site). In some aspects, the alternating electrical field can be in a single direction or multiple directional. In some aspects, alternating electric fields can be delivered through two pairs of transducer arrays that generate perpendicular fields within the treated heart. For example, for the Optune™ system (an alternating electric fields delivery system) one pair of electrodes is located to the left and right (LR) of the infection site, and the other pair of electrodes is located anterior and posterior (AP) to the infection site. Cycling the field between these two directions (i.e., LR and AP) ensures that a maximal range of cell orientations is targeted.

In-vivo and in-vitro studies show that the efficacy of alternating electric fields therapy increases as the intensity of the electrical field increases. Therefore, optimizing array placement on the patient's chest to increase the intensity in the desired region of the heart can be performed with the Optune system. Array placement optimization may be performed by “rule of thumb” (e.g., placing the arrays on the chest as close to the desired region of the target site (e.g. tumor or lymph node as possible), measurements describing the geometry of the lymph node and/or tumor location. Measurements used as input may be derived from imaging data. Imaging data is intended to include any type of visual data. In certain implementations, image data may include 3D data obtained from or generated by a 3D scanner (e.g., point cloud data). Optimization can rely on an understanding of how the electrical field distributes within the target site as a function of the positions of the array and, in some aspects, take account for variations in the electrical property distributions within the target site of different patients.

The term “subject” refers to the target of administration, e.g. an animal. Thus, the subject of the disclosed methods can be a vertebrate, such as a mammal. For example, the subject can be a human. The term does not denote a particular age or sex. Subject can be used interchangeably with “individual” or “patient.” For example, the subject of administration can mean the recipient of the alternating electrical field.

By “treat” is meant to administer or apply a therapeutic, such as alternating electric fields, to a subject, such as a human or other mammal (for example, an animal model), that has an infection or has an increased susceptibility for developing an infection, in order to prevent or delay a worsening of the effects of the infection, or to partially or fully reverse the effects of the infection.

By “prevent” is meant to minimize the chance that a subject who has an increased susceptibility for developing an infection will develop an infection.

As used herein, the terms “administering” and “administration” refer to any method of providing a therapeutic, such as an anti-inflammatory, to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration so as to treat a subject. In some aspects, administering comprises exposing. Thus, in some aspects, exposing an infection site to alternating electrical fields means administering alternating electrical fields to the infection site.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods

The primary function of CD8+ (or cytotoxic) T cells is to recognize and kill infected cells and cancer cells. The establishment of a pool of memory CD8+ T cells is the goal of T-cell vaccination strategies, and understanding how to modulate their function is critical for vaccine development and immunotherapies. The mere increase in the number of CD8+ T cells, thus the proliferation of CD8+ T cells, can provide an array of beneficial effects.

In some aspects of the methods, a plurality of electrodes can be positioned in or on a subject's body positioned with respect to the target site so that application of an AC voltage between the plurality of electrodes will impose an alternating electric field through tissue that is being infected or in need of a pro-inflammatory response in the target site; and applying an AC voltage between the plurality of electrodes for an interval of time, such that an alternating electric field is imposed through the tissue for the interval of time. The alternating electric field has a frequency and a field strength such that when the alternating electric field is imposed in the tissue for the interval of time, the alternating electric field increases proliferation of T cells in the tissue to an extent that reduces damage that is caused by inflammation.

### 1. Methods of Increasing Proliferation of Stimulated CD8+ T

Disclosed are methods of increasing proliferation of CD8+ T cells comprising exposing a target site to an alternating electric field for a period of time, the alternating electric field having a frequency and field strength, wherein the frequency and field strength of the alternating electric field increases proliferation of CD8+ T cells at the target site.

In some aspects, the target site comprises stimulated CD8+ T cells. In some aspects, the stimulated CD8+ T cells were stimulated by recognition of a class I peptide on an antigen presenting cell. In some aspects, the class I peptide can be a peptide from a viral, bacterial, fungal, parasitic, or tumor antigen.

In some aspects, the target site is a site of inflammation. In some aspects, a site of inflammation can be any location in a subject undergoing an inflammatory response. For example, a site of inflammation can include, but is not limited to, an area on a subject that has redness or heat, an accumulation of fluid, or pain. In some aspects, the site of inflammation can be a burn site, an infection site, or an amputation site. In some aspects, a burn site can be any location on a subject that has been affected by burns. In some aspects, an infection site can be any location on or in a subject wherein the tissue in that location has been invaded by an infectious agent. An infectious agent can be, but is not limited to, viruses, bacteria, fungi, parasites, or arthropods. Thus, in some aspects, an infection site can be a viral infection site, meaning an infection site caused by a virus. In some aspects, a viral infection site can be a Pneumonia viral infection site, Meningitis viral infection site, or Herpes zoster viral infection site, meaning a viral infection site caused by Pneumonia, Meningitis, or Herpes zoster, respectively. In some aspects, an amputation site can be any location wherein an organ or limb has been removed.

In some aspects, the target site is a site comprising a draining lymph node. In some aspects, the draining lymph node is near or adjacent to a site of inflammation. In some aspects, the draining lymph node is a site of inflammation. In some aspects, the site of a draining lymph node is near or adjacent to a primary tumor.

In some aspects, the increased proliferation of CD8+ T cells results in more CD8+ T cells capable of cytotoxic activity. In some aspects, these proliferated CD8+ T cells can then fight off infection or tumors by killing infected cells or tumor cells.

### 2. Methods of Generating a Pro-Inflammatory Response

Disclosed are methods of generating a pro-inflammatory response in a target site comprising exposing the target site to an alternating electric field for a period of time, the alternating electric field having a frequency and field strength, wherein the frequency and field strength of the alternating electric field generates a pro-inflammatory response at the target site.

In some aspects, a pro-inflammatory response comprises one or more cytokines. For example, in some aspects, a pro-inflammatory response can comprise one or more cytokines. In some aspects, the one or more aspects can be IFN-γ, TNF-α, IL1, IL18, or granulocyte-macrophage colony stimulating factor (GM-CSF).

In some aspects, the target site comprises stimulated CD8+ T cells.

In some aspects, the target site is a site of inflammation. In some aspects, a site of inflammation can be any location in a subject undergoing an inflammatory response. For example, a site of inflammation can include, but is not limited to, an area on a subject that has redness or heat, an accumulation of fluid, or pain. In some aspects, the site of inflammation can be a burn site, an infection site, or an amputation site. In some aspects, a burn site can be any location on a subject that has been affected by burns. In some aspects, an infection site can be any location on or in a subject wherein the tissue in that location has been invaded by an infectious agent. An infectious agent can be, but is not limited to, viruses, bacteria, fungi, parasites, or arthropods. Thus, in some aspects, an infection site can be a viral infection site, meaning an infection site caused by a virus. In some aspects, a viral infection site can be a Pneumonia viral infection site, Meningitis viral infection site, or Herpes zoster viral infection site, meaning an viral infection site caused by Pneumonia, Meningitis, or Herpes zoster, respectively. In some aspects, an amputation site can be any location wherein an organ or limb has been removed.

In some aspects, the target site is a site comprising a draining lymph node. In some aspects, the draining lymph node is near or adjacent to a site of inflammation. In some aspects, the draining lymph node is a site of inflammation. In some aspects, the site of a draining lymph node is near or adjacent to a primary tumor.

### 3. Methods of Increasing Proliferation of CD8+ T Cells

Disclosed are methods of increasing proliferation of CD8+ T cells comprising exposing CD8+ T cells to an alternating electric field for a period of time, the alternating electric field having a frequency and field strength, wherein the frequency and field strength of the alternating electric field increases proliferation of CD8+ T cells. In some aspects, exposing CD8+ T cells to an alternating electric field comprises exposing lymphatic tissue to an alternating electric field wherein the lymphatic tissue comprises CD8+ T cells. Thus, exposing CD8+ T cells to an alternating electric field can be direct or indirect exposure.

In some aspects, the CD8+ T cells are stimulated CD8+ T cells. In some aspects, the CD8+ T cells are stimulated in vivo. In some aspects, the CD8+ T cells are stimulated in vitro.

In some aspects, the CD8+ T cells are stimulated by recognition by recognition of a class I peptide on an antigen presenting cell. In some aspects, the class I peptide can be a peptide from a viral, bacterial, fungal, parasitic, or tumor antigen. In some aspects, the CD8+ T cells are stimulated by a mitogen, such as but not limited to PHA, an antibody, Concanavalin A, or wheat germ agglutinin.

In some aspects, the CD8+ T cells are in a subject.

In some aspects, the CD8+ T cells are exposed to the alternating electrical field in vitro. In some aspects, the CD8+ T cells are exposed to the alternating electric field through a container suitable for cell culture. In some aspects, a container suitable for cell culture can be a tissue culture flask or a petri dish.

Also disclosed are methods of increasing proliferation of CD8+ T cells comprising exposing CD8+ T cells to an alternating electric field for a period of time, the alternating electric field having a frequency and field strength, wherein the frequency and field strength of the alternating electric field increases proliferation of CD8+ T cells, further comprising a step of administering the CD8+ T cells exposed to the alternating electric field into a subject. Thus, this method can be an ex vivo therapeutic treatment. In some aspects, the subject can have an infection, cancer, a burn or has recently undergone an organ amputation.

Disclosed are methods for inducing a population of CD8⁺ T cells to proliferate, comprising activating a population of T cells; and exposing the population of T cells to an alternating electric field for a period of time, the alternating electric field having a frequency and field strength, wherein the activating and exposing steps thereby induce proliferation of the CD8+ T cells.

In some aspects, the population of T cells can be activated in vitro. In some aspects, in vitro activation includes contacting the population of T cells with a mitogen. In some aspects, the mitogen can be, but is not limited to, PHA, an antibody, Concanavalin A, or wheat germ agglutinin. In some aspects, the T cells are activated by recognition by recognition of a class I peptide on an antigen presenting cell. In some aspects, the class I peptide can be a peptide from a viral, bacterial, fungal, parasitic, or tumor antigen.

### 4. Methods of Treating

Disclosed are methods of treating a subject in need of a CD8+ T cell response comprising applying an alternating electric field to a target site of the subject for a period of time, the alternating electric field having a frequency and field strength, wherein the target site comprises one or more CD8+ T cells.

In some aspects, a subject in need of a CD8+ T cell response is a subject having an infection, cancer, a burn, or having recently undergone an organ amputation. In some aspects, the infection can be a viral infection. In some aspects, the viral infection can be from Pneumonia, Meningitis, or Herpes zoster.

In some aspects, the target site is a site of inflammation. In some aspects, a site of inflammation can be any location in a subject undergoing an inflammatory response. For example, a site of inflammation can include, but is not limited to, an area on a subject that has redness or heat, an accumulation of fluid, or pain. In some aspects, the site of inflammation can be a burn site, an infection site, or an amputation site. In some aspects, a burn site can be any location on a subject that has been affected by burns. In some aspects, applying the electrical field to a burn site regenerates skin growth at the burn site. In some aspects, an infection site can be any location on or in a subject wherein the tissue in that location has been invaded by an infectious agent. An infectious agent can be, but is not limited to, viruses, bacteria, fungi, parasites, or arthropods. Thus, in some aspects, an infection site can be a viral infection site, meaning an infection site caused by a virus. In some aspects, a viral infection site can be a Pneumonia viral infection site, Meningitis viral infection site, or Herpes zoster viral infection site, meaning an viral infection site caused by Pneumonia, Meningitis, or Herpes zoster, respectively. In some aspects, an amputation site can be any location wherein an organ or limb has been removed.

In some aspects, the target site is a site comprising a draining lymph node. In some aspects, the draining lymph node is near or adjacent to a site of inflammation. In some aspects, the draining lymph node is a site of inflammation. In some aspects, the site of a draining lymph node is near or adjacent to a primary tumor.

In some aspects, the target site is a tumor.

In some aspects, the target site comprises stimulated CD8+ T cells. In some aspects, the stimulated CD8+ T cells were stimulated by recognition of a class I peptide on an antigen presenting cell. In some aspects, the class I peptide can be a peptide from a viral, bacterial, fungal, parasitic, or tumor antigen.

In some aspects, the disclosed methods of treating further comprise administering a therapeutic that alters the immune system. In some aspects, a therapeutic that alters the immune system can be an anti-inflammatory agent, a pro-inflammatory agent, cytokines, antibodies, proteins, or nucleic acids.

### C. Alternating Electric Fields

The methods disclosed herein comprise alternating electric fields. In some aspects, the alternating electric field used in the methods disclosed herein is a tumor-treating field. In some aspects, the alternating electric field can vary dependent on the specific site or condition to which the alternating electric field is applied. In some aspects, the alternating electric field can be applied through one or more electrodes placed on the subject's body. In some aspects, the alternating electric field can be applied through one or more electrodes placed on a container suitable for cell culture. In some aspects, there can be two or more pairs of electrodes. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

The same frequency that is used in the Optune® system to treat glioblastoma (i.e., 200 kHz) may also be used to treat an infection by increasing the proliferation of T cells, as described above. But in alternative embodiments, a different frequency may be used. For example, the frequency of the alternating electric fields can be 150 kHz. In some aspects, the frequency of the alternating electric fields can be 200 kHz or 300 kHz. In some aspects, the frequency of the alternating electric fields can be 120-170 kHz. The frequency of the alternating electric fields can also be, but is not limited to, about 150 kHz, between 50 and 500 kHz, between 100 and 500 kHz, between 25 kHz and 1 MHz, between 50 and 190 kHz, between 25 and 190 kHz, between 150 and 300 kHz, or between 210 and 400 kHz. In some aspects, the frequency of the alternating electric fields can be electric fields at 50 kHz, 100 kHz, 150 kHz, 200 kHz, 300 kHz, 400 kHz, 500 kHz, or any frequency between.

In some embodiments the frequency of the alternating electric field is from about 100 kHz to about 200 kHz, from about 150 kHz to about 250 kHz, and may be around 300 kHz.

In some aspects, the field strength of the alternating electric fields can be between 1 and 4 V/cm RMS. In some aspects, different field strengths can be used (e.g., between 0.1 and 10 V/cm). In some aspects, the field strength can be 1.75 V/cm RMS. In some embodiments the field strength is at least 1 V/cm. In other embodiments combinations of field strengths are applied, for example combining two or more frequencies at the same time, and/or applying two or more frequencies at different times.

In some aspects, the alternating electric fields can be applied for a variety of different intervals ranging from 0.5 hours to 72 hours. In some aspects, the alternating electric fields can be applied anywhere from 1 to 10 days. In some aspects, the alternating electric fields can be applied anywhere from days to weeks to months. In some aspects, the alternating electric fields can be applied for at least 3 days. In some aspects, the alternating electric fields can be applied for 1 week, 2 weeks, 3 weeks, or 4 weeks. In some aspects, a different duration can be used (e.g., between 0.5 hours and 14 days). In some aspects, application of the alternating electric fields can be repeated periodically. For example, the alternating electric fields can be applied every day for a two hour duration.

In some aspects, the exposure may last for at least 6 hours, at least 12 hours, at least 24 hours, at least 36 hours, at least 48 hours, or at least 72 hours or more. Thus, in some aspects, application of the alternating electric fields can be continuous.

The orientation of the alternating electric currents may be switched at one second intervals between two different orientations by applying AC voltages between two different sets of electrodes, as done in the Optune® system. But in alternative embodiments, the orientation of the alternating electric currents can be switched at a faster rate (e.g., at intervals between 1 and 1000 ms) or at a slower rate (e.g., at intervals between 1 and 100 seconds). In other alternative embodiments, the electrodes need not be arranged in pairs. See, for example, the electrode positioning described in U.S. Pat. No. 7,565,205, which is incorporated herein by reference. In other alternative embodiments, the orientation of the field need not be switched at all, in which case only a single pair of electrodes is required.

In some aspects, the electrodes are capacitively coupled to the subject's body (e.g., by using electrodes that include a conductive plate and also have a dielectric layer disposed between the conductive plate and the subject's body). But in alternative embodiments, the dielectric layer may be omitted, in which case the conductive plates would make direct contact with the subject's body.

Optionally, thermal sensors may be included at the electrodes, and the AC voltage generator can be configured to decrease the amplitude of the AC voltages that are applied to the electrodes if the sensed temperature at the electrodes gets too high.

In some aspects, one or more additional pairs of electrodes may be added and included in the sequence. In other embodiments, the field is only imposed in the target region with a single orientation, in which case the alternating sequence described above may be replaced with a continuous AC signal that is applied to a single set of electrodes (e.g., positioned on opposite sides of the target region).

### 1. Positioning of Electrodes

Positioning of electrodes can help provide the alternating electric currents to the target site.

In some aspects, the positioning comprises positioning a first set of electrodes in or on the subject's body and positioning a second set of electrodes in or on the subject's body. The first set of electrodes is positioned with respect to the target site so that application of an AC voltage between the electrodes of the first set will impose an alternating electric field with a first orientation through the tissue that is being infected in the target site. The second set of electrodes is positioned with respect to the target site so that application of an AC voltage between the electrodes of the second set will impose an alternating electric field with a second orientation through the tissue. The first orientation and the second orientation are different. The applying comprises repeating, in an alternating sequence, (a) applying a first AC voltage between the electrodes of the first set, such that an alternating electric field with the first orientation is imposed through the tissue and (b) applying a second AC voltage between the electrodes of the second set, such that an alternating electric field with the second orientation is imposed through the tissue. The alternating electric field with the first orientation has a frequency and a field strength such that when the alternating electric field with the first orientation is imposed in the tissue, the alternating electric field with the first orientation increases proliferation of T cells in the tissue. The alternating electric field with the second orientation has a frequency and a field strength such that when the alternating electric field with the second orientation is imposed in the tissue, the alternating electric field with the second orientation increases proliferation of T cells in the tissue. The increased proliferation of T cells in the tissue helps fight an infection in the target site.

Optionally, in some aspects, the first and second sets of electrodes may also be positioned with respect to the subject's body so that the alternating electric fields with the first and second orientations are also imposed in at least one draining lymph node associated with the tissue that is being attacked. Optionally, in the instances of the first method described in the previous paragraph, the first orientation is offset from the second orientation by at least 60°.

In some aspects, a plurality of electrodes are positioned in or on a subject's body positioned with respect to at least one draining lymph node associated with the tissue that is being attacked so that application of an AC voltage between the plurality of electrodes will impose an alternating electric field through the at least one draining lymph node; and applying an AC voltage between the plurality of electrodes for an interval of time, such that an alternating electric field is imposed through the at least one draining lymph node for the interval of time. The alternating electric field has a frequency and a field strength such that when the alternating electric field is imposed in the at least one draining lymph node for the interval of time, the alternating electric field increases proliferation of T cells in the at least one draining lymph node to an extent that causes a pro-inflammatory response to fight off an infection.

In some aspects, positioning comprises positioning a first set of electrodes in or on the subject's body and positioning a second set of electrodes in or on the subject's body. The first set of electrodes is positioned with respect to the at least one draining lymph node associated with the tissue that is being attacked so that application of an AC voltage between the electrodes of the first set will impose an alternating electric field with a first orientation through the at least one draining lymph node, and the second set of electrodes is positioned with respect to the at least one draining lymph node so that application of an AC voltage between the electrodes of the second set will impose an alternating electric field with a second orientation through the at least one draining lymph node. The first orientation and the second orientation are different. The applying comprises repeating, in an alternating sequence, (a) applying a first AC voltage between the electrodes of the first set, such that an alternating electric field with the first orientation is imposed through the at least one draining lymph node and (b) applying a second AC voltage between the electrodes of the second set, such that an alternating electric field with the second orientation is imposed through the at least one draining lymph node. The alternating electric field with the first orientation has a frequency and a field strength such that when the alternating electric field with the first orientation is imposed in the at least one draining lymph node, the alternating electric field with the first orientation increases proliferation of T cells in the at least one draining lymph node. The alternating electric field with the second orientation has a frequency and a field strength such that when the alternating electric field with the second orientation is imposed in the at least one draining lymph node, the alternating electric field with the second orientation increases proliferation of T cells in the at least one draining lymph node.

In the embodiments, a system that is similar to the Optune® system for treating tumors with TTFields is used to treat infections or other circumstances where a pro-inflammatory response is needed instead of treating a tumor. Although use of the Optune® system for treating glioblastoma is well-understood by persons skilled in the relevant arts, it will be described here briefly for completeness. Four arrays of capacitively coupled electrodes (also called “transducer arrays”) are positioned on the subject' shaved head (e.g., one on the front, one on the back, one on the right side, and one on the left side). An AC voltage generator applies an AC voltage at 200 kHz between the front/back pair of electrode arrays for one second, then applies an AC voltage at the same frequency between the right/left pair of electrode arrays for one second, and repeats this two-step sequence for the duration of the treatment. This induces TTFields in the first and second orientations through the subject's brain in an alternating sequence. The electrode arrays are positioned so that the first orientation and the second direction are offset by a significant amount (e.g., at least 60°, or at least 80°).

In some aspects, all the electrodes are positioned on the subject's body; in other aspects, all the electrodes may be implanted in the subject's body (e.g., just beneath the subject's skin, or in the vicinity of the organ being treated); and in other embodiments, some of the electrodes are positioned on the subject's skin and the rest of the electrodes are implanted in the subject's body.

### D. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example, disclosed are kits for imaging and/or treating. In some aspects, the kits comprise equipment for applying alternating electrical fields.

Also disclosed are kits comprising a system or equipment for administering alternating electrical fields and one or more of the disclosed second therapeutics, such as, an anti-inflammatory, anti-viral, anti-bacterial, or anti-cancer drug.

### EXAMPLES

Disclosed herein, the inovitro® tissue culturing system was used to evaluate the effect of TTFields on T cells. TTFields' effects on T cell proliferation, viability and select pivotal anti-tumoral functions were examined using both healthy donor blood and TILs from resected GBM samples, representing a solid tumor currently treated by TTFields as common practice. Direct cytotoxicity was evaluated using a CAR-T cell-based system. Immunohistochemical analysis and comparative transcriptomic evaluation on GBM samples from patients before and after TTFields treatments was used to assess treatment effects on T cell infiltration and on immune gene expression.

### 1. Materials and Methods

### i. Cells and Tissue Culture

PBMC were produced from leukocyte-enriched fractions from healthy blood donations obtained from the Israeli blood bank. Tissue culture was performed in X-VIVO15 medium (Lonza Basel, Switzerland) supplemented with 1:5000 Benzonase (Novagen, Billerica, Mass.).

Human GBM viable single-cell suspensions were produced by dissociating fresh tumor samples using neutral protease from Clostridium histolyticum (AMSBio, Abingdon, UK). Institutional approval was received for use of blood donations and GBM samples (0408-10-TLV).

CAG multiple myeloma cells were obtained from the ATCC (Manassas, Va.) and maintained in RPMI 1640 (Gibco, Grand Island, N.Y.), 10% FBS, 1% sodium pyruvate, 1% glutamine and 1% penicillin/streptomycin (Biological Industries, Israel). HER2-expressing CAG cells were generated by transfection (jetPRIME, Polyplus, France) with human HER2 and G418 resistance genes. HER2-CAG cells were cultured in the CAG cell culture medium supplemented with 0.5 mg/ml G418 (Gibco). T cells expressing an anti-HER2 Chimeric Antigen Receptor (CAR) were generated by retroviral transduction of healthy donor PBMC with a vector encoding the N29 anti-Her2 CAR and GFP.

### ii. TTFields Application In-Vitro

Cell culturing under TTFields conditions was conducted using the inovitro® TTFields system according to manufacturer's instructions unless described otherwise. Cells were directly seeded in the inovitro culture dishes and incubated either in a standard incubator or on baseplates generating an electric field in a heating/cooling incubator (Binder, Tuttlingen, Germany). Application of TTFields to the inovitro culture dishes creates excess heat, which is offset by incubation in an ambient temperature below 37° C. This accelerates evaporation of the media, requiring changing them daily for adherent cell cultures. Since this is impractical for non-adherent cultures, an alternative protocol was developed based on replenishing the media with DDW. The electric parameters used in all assays matched those used in clinical GBM therapy. Field frequency was set to 200 kHz. Field intensity was set at 2-2.5V/cm (peak-to-peak), which is within the range found to optimally inhibit GBM cell growth and as used in GBM patients.

### iii. T cell Activation and Polyfunctionality Assays

PBMC (3×10⁶) and dissociated single-cells from GBM samples were CFSE-stained and cultured as described above. Five hours before harvesting, the cultures was supplemented with fluorochrome-conjugated CD107a antibody, 0.07% Golgistop (BD Biosciences) and 0.1% Brefeldin A (Sigma). Cells were then collected, stained with a viability dye and then extracellularly for CD8a, CD14, CD19 and PD1, and intracellularly for CD3, CD4 and IFNγ.

### iv. Multicolor Flow Cytometry

Multicolor flow cytometry was performed using a Canto-II flow cytometer (BD Biosciences, Franklin Lakes, N.J.). Staining was performed in the dark and at room temperature. The staining antibodies were: CD3, CD4 and CD19 from eBioscience (San Diego, Calif.), CD107a, CD14, PD1 and IFNγ from Biolegend (San Diego, Calif.) and CD8a from BD Biosciences. Data analysis was performed using FlowJo (FlowJo LLC, Ashland, Oreg.).

### v. CFSE Staining

PBMC or GBM cell suspensions (1-5×10⁷ cells/ml) were stained with CFSE (Thermo Fisher Scientific, Waltham, Mass.) by incubation in PBS^{-/-} with 2 μM CFSE and 1.25% FCS (10 minutes, room temperature). The cells were then washed twice with PBS, 2.5% FCS, counted using trypan blue (Sigma, St. Louis, Mo.) and used for subsequent assays.

### vi. Viability Assay

CFSE-stained PBMCs (3×10⁶ cells/dish) were seeded on inovitro dishes in 2 ml X-VIVO15 and either stimulated with 1:100 phytohemagglutinin (PHA, Gibco) or left untreated. The cells were incubated for 3.5 days under either TTFields or standard conditions. Cells were then collected, stained with ViViD amine viability dye (Thermo Fischer) and then stained extracellularly for CD8a, CD14 and CD19. CD3 and CD4 were stained intracellularly using the Fix/Perm Kit (BD Biosciences)²⁵ as T cell activation drives their internalization. The cells were then fixed at 1% formaldehyde (Electron Microscopy Sciences, Hatfield, Pa.) and analyzed by flow cytometry.

To enable inclusion of dead cells in this assay, auto-fluorescent debris (particles with medium-to-high signals on all channels that can be mistaken for dead cells) were gated out based on a higher CFSE signal and a lower side-scatter signal compared to T cells. Viability rates were calculated as live/total cells. The cells were categorized as live (ViViD^{low}) or dead (ViViD^{high}), and as proliferating (CFSE^{low}) or non-proliferating (CFSE^{high}).

### vii. In-Vitro Cytotoxicity Assay

Target cells (CAG-HER2 or CAG) and effector anti-HER2 human CAR T cells were co-cultured for 8 hours at varying effector:target ratios in standard or TTFields culture conditions. Cultures were stained with CD3 and CD138 (myeloma marker) and propidium iodide (PI), staining dead cells (Sigma) and then analyzed by flow cytometry. Cell death rates in ‘target-only’ were subtracted from those in matching ‘effector:target’. The unspecific killing of parental CAG (non-HER2) was subtracted from its corresponding CAG-Her2 sample.

### viii. Statistical Analysis

In-vitro quantitative data are presented as mean±standard error. Statistical significance was determined by paired, two-tailed Student's t-test and noted where significant (*p<0.05, ***p<0.005). All in-vitro experiments were repeated at least three times.

### ix. Tissue Immunohistochemistry and Analysis

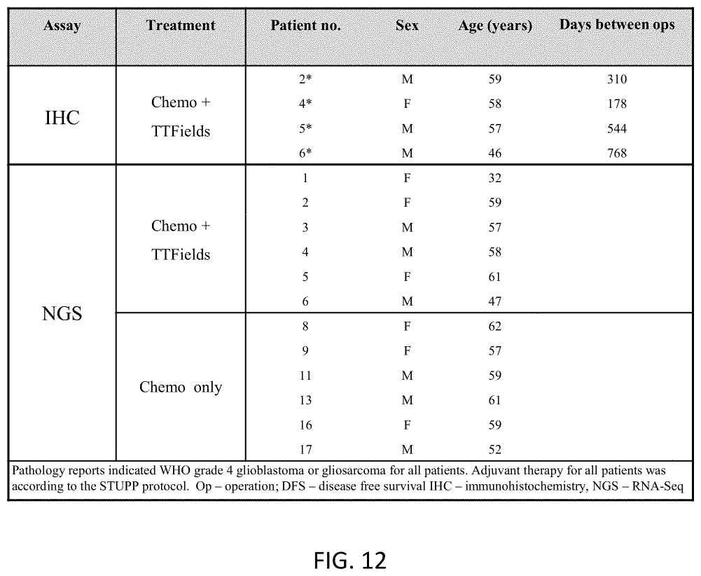

Tumor samples were obtained from four GBM patients before and after TTFields treatment (patient data in FIG. 12). Tissue slides were immunohistochemically stained by hospital pathology services for CD3, CD4 and CD8 by the DAB staining procedure. Tissue slides were scanned and then analyzed with the Aperio Imagescope program. Stained (i.e., brown colored) nucleated cells (i.e., blue center) were counted in 4-6 representative 0.4 mm² fields in each slide. The cell counts were then averaged and normalized as cell count/mm².

### x. Tissue Processing for RNA-Seq and Library Construction

Tumor tissues from twelve newly diagnosed GBM patients were obtained before and after treatment with standard chemoradiation (six controls) or with TTFields+chemoradiation (six TTFields). RNA sequencing was performed on these samples.

### xi. Transcriptomic Analysis

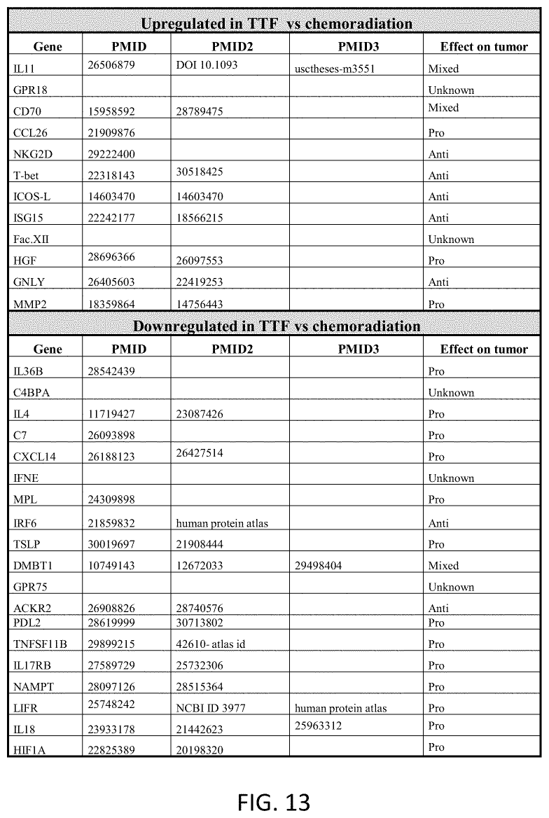

RNA-Seq data were analyzed with DESeq2 software. Gene expression was compared on a broad list of 712 immune-related genes. The difference in expression before and after treatments was calculated per patient, and then averaged as net treatment effect per group. Fold-changes of net treatment effects above 2 or below 0.5, with a Benjamini-Hochberg multiple-comparison-corrected p value <0.1 were defined as significant. The pro-tumoral/anti-tumoral/mixed activity of each significantly altered gene was determined using published literature (FIG. 13).

### xii. Cells and Tissue Culture

Leukocyte-enriched fractions from healthy blood donations were obtained from the Israeli blood bank. Peripheral blood mononuclear cells (PBMC) were isolated by buffy coat method, using Lymphoprep (STEMCELL technologies, Vancouver, Canada). Frozen PBMC aliquots were thawed in X-VIVO15 defined medium (Lonza Basel, Switzerland) with 1:5000 Benzonase (Novagen, Billerica, Mass.).

Human GBM single-cell suspensions were produced by dissociating freshly resected samples using neutral protease from Clostridium histolyticum (AMSBio, Abingdon, UK)[21]. The dissociated tumor cells were freshly assayed.

### xiii. TTFields Application In-Vitro

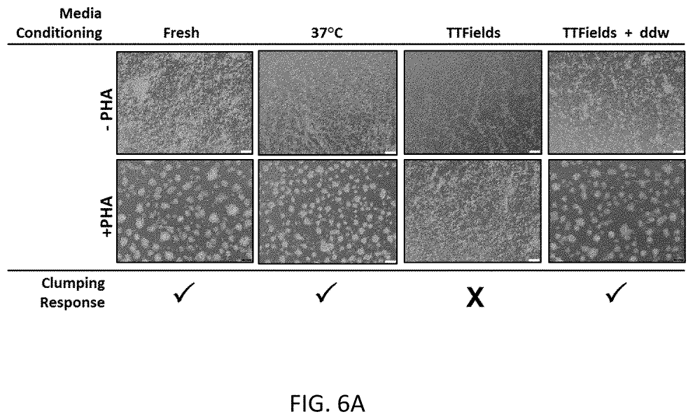

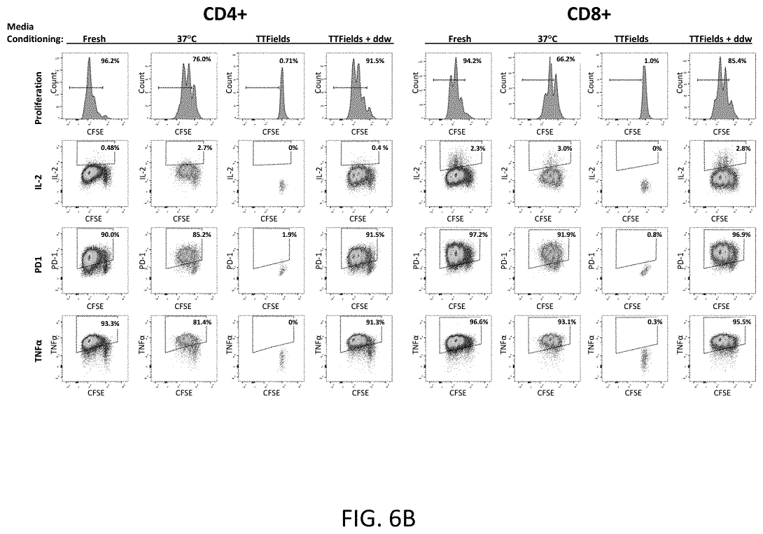

The temperature difference between the incubator and the dish accelerates media evaporation. This was addressed by daily replenishment of the evaporated volumes with double distilled water (FIG. 6).

### xiv. In-Vitro Cytotoxicity Assay

Target cells (CAG-HER2 or CAG) and effector anti-HER2 human CAR T cells were washed with RPMI medium, and then co-seeded at varying effector:target ratios in Inovitro dishes supplemented with 1:5000 Benzonase to reduce split DNA/RNA-related clumping. Cells were incubated under normal or TTFields conditions for 8 h, collected, washed with FACS buffer (PBS^{-/-}, 2 mM EDTA, 2% FBS) and stained with CD3 and CD138 (myeloma marker) antibodies for 20 min. Cells were then washed, brought to a final volume of 300 μl in FACS buffer and placed on ice. Samples were stained with 1:2000 propidium iodide (PI) (Sigma) for 2 min and analyzed by flow cytometry. Cell death rates of ‘target-only’ samples (“baseline”) were subtracted from the rates measured in matching ‘effector:target’ samples. The killing rate in each CAG (non HER2) sample was subtracted as unspecific killing from its corresponding CAG-Her2 sample.

### xv. Tissue Processing for RNA-Seq and Library Construction

Tumor tissues from twelve newly diagnosed GBM patients were obtained before and after treatment with standard chemoradiation (6 controls) or with TTFields+chemoradiation (6 TTFields). Tissues were flash-frozen using liquid nitrogen upon resection. Processing and RNA extraction were performed with the PerfectPure RNA tissue kit (5 prime GmbH, Hilden, Germany). RNA integrity was assessed by electrophoresis. Illumina TruSeq® RNA Library Prep v2 was used for sample preparation, generating mRNA-based libraries from total RNA input. Indexed samples were sequenced, in single read mode, using the Illumina HiSeq 2500.

### xvi. Transcriptomic Analysis

Raw data were analyzed using DESeq2 software. Differential gene expression analysis was calculated using the reads per kilobase million (RPKM) values of chemoradiation and TTFields treatment groups. Statistical analysis was performed using the negative binomial generalized linear model. The difference between expression before and after treatment was derived separately for each individual and the average net treatment effect was calculated for each treatment group. The difference between treatment effects was represented as fold change between the average net effects of TTFields and control. The Benjamini-Hochberg method was used to correct fold change p values for multiple comparisons. A significant difference between TTFields and control treatment effects was defined as fold change >2 or <0.5 with a corrected p value <0.1. A list of 712 genes related to immune activity was compiled using the general literature, the Nanostring “nCounter® PanCancer immune profiling panel” and the ThermoFischer “Oncomine™ immune response” gene lists. The list surveys all chemokines, cytokines and their receptors, transcription factors, immune checkpoint-related molecules and other immune-related genes. The pro-tumoral/anti-tumoral/mixed activity designation of each significantly altered gene was determined according to the general literature (FIG. 13).

Examining the significance of the association (contingency) between pro-tumoral to anti-tumoral genes significantly up or down regulated by TTFields or by standardly-treated patients was calculated using the Fisher's exact method.

### 2. Results

### i. TTFields Reduce the Viability of Proliferating T Cells

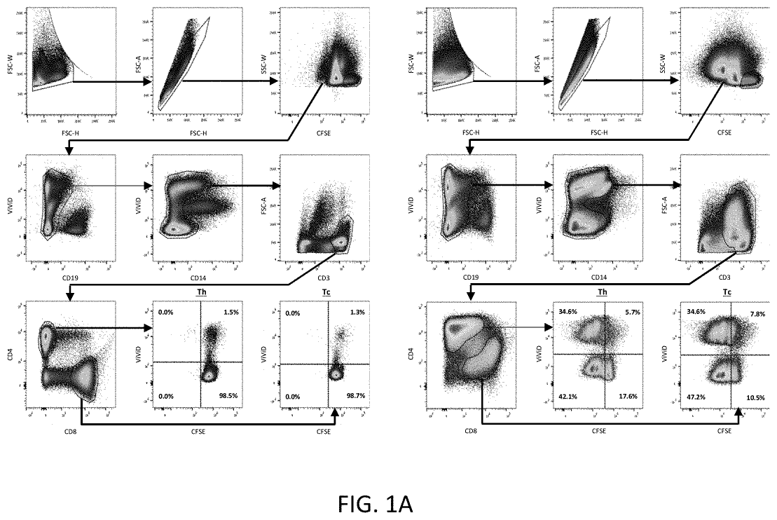

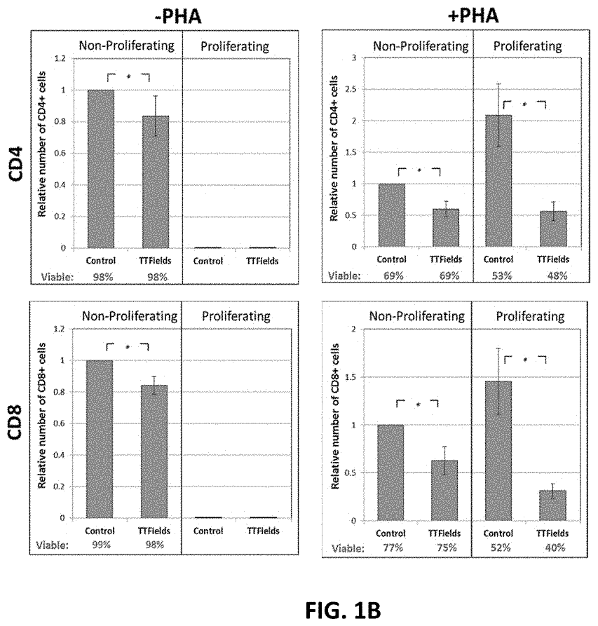

TTFields exert a cytostatic and cytotoxic effect on proliferating tumor cells. To evaluate T cell viability under TTFields, PBMCs were stained with CFSE and stimulated with PHA to induce proliferation. PHA was selected since it activates T cells via their T cell receptor, thereby simulating physiological activation that requires signal transduction, organelle redistribution and actin cytoskeletal dynamics. It is important to note that PHA stimulation is robust, leading to both activation and proliferation as well as to activation-induced cell death. This affects both cell numbers and the viability rate. The FACS gating strategy identified CD4+ and CD8+ T cells, gating out B cells, monocytes, and autofluorescent debris and then evaluated proliferating/non-proliferating cells according to their viability (FIG. 1A).

No proliferation was detected in the PHA-unstimulated samples, and the number of cells in the TTFields samples was approximately 85% of standardly incubated controls (FIG. 1B, left, P=0.01). No changes were detected in the viability rates (% live/dead) of the unstimulated cultures in TTFields versus controls, suggesting that TTFields have from low to no effect on the viability of unstimulated T cells.

In the PHA-stimulated samples, PHA drove part of the T cells to proliferate and affected both cell number and viability. The number of non-proliferating (CFSEhigh) T cells in the stimulated TTFields-cultured samples was approximately 65% that of the controls (p=0.015 and p=0.03 for CD4+ and CD8+ cells, respectively). The viability rates remained unchanged (FIG. 1B, right). In the same samples, the number of proliferating T cells in TTFields-cultured samples substantially decreased to approximately 25% of controls (p=0.009 and p=0.01 for CD4+ and CD8+, respectively) along with a decline in the viability rates, especially for the CD8+ T cells (52% in the controls versus 40% in TTFields). These results align with the known MOA of TTFields, i.e., marked inhibition and cytotoxic effect on proliferating cells and low impact on non-proliferating cells.

### ii. Key Anti-Tumoral Functions in Stimulated Blood T Cells are Unaffected by TTFields

Many immune-related cellular processes rely on cytoskeletal dynamics or on vesicular transport that may conceivably be perturbed by TTFields. These processes include cytokine secretion, mobilization of surface molecules and cytotoxic-granule degranulation. To investigate whether key antitumoral cellular functions are affected by TTFields, proliferation (CFSE), activation/exhaustion (PD1), IFNγ secretion and cytotoxic granule degranulation (CD107a 29) were simultaneously monitored at single-cell resolution.

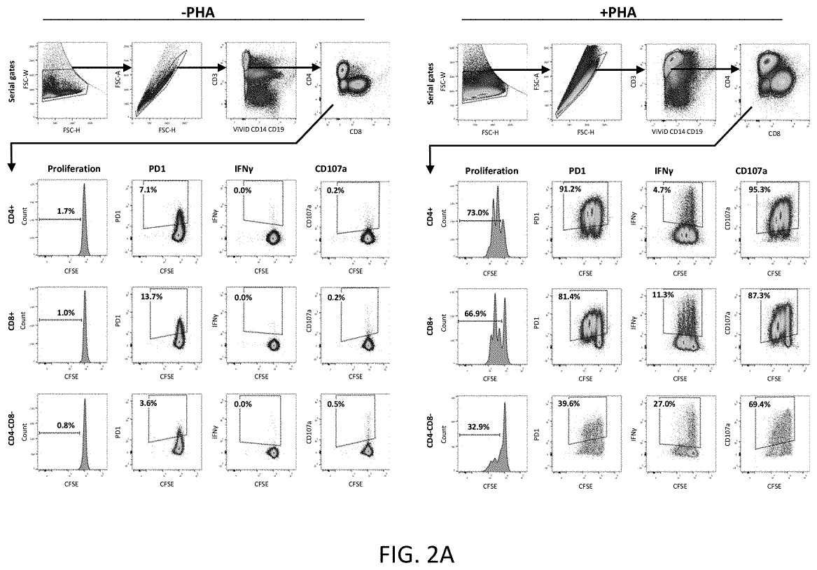

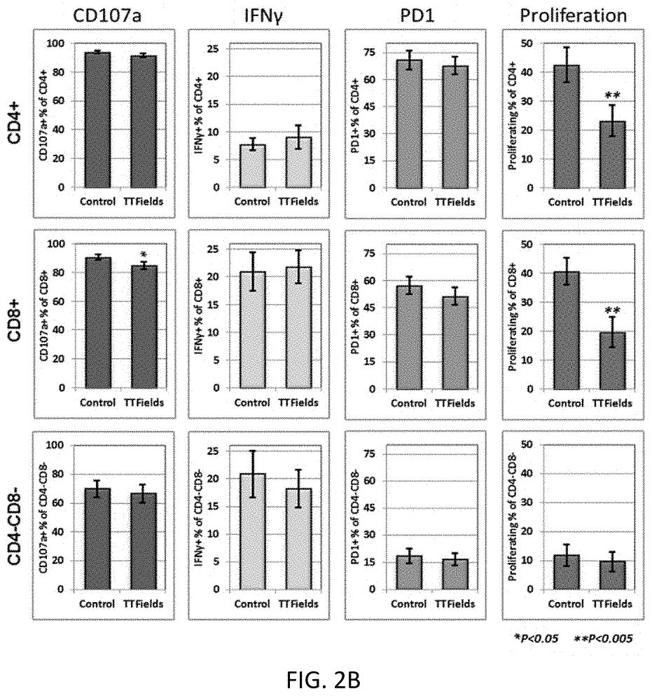

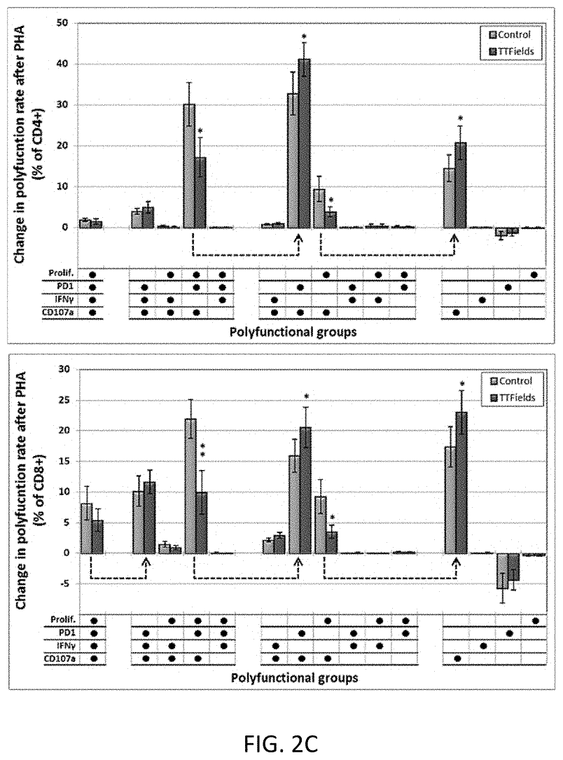

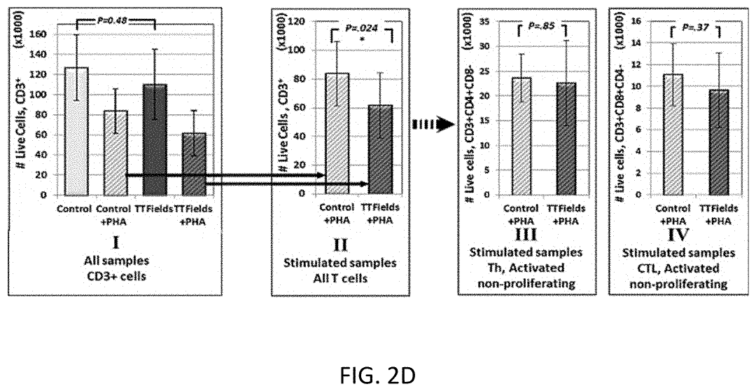

FACS gating strategy (FIG. 2A) identified CD4+ and CD8+ T cells, alongside CD3+CD14-CD19-CD4-CD8- cells (which consisted of >90% γδ T-cells, not shown). Other than an approximately 50% reduction in the fraction of proliferating CD4+ or CD8+ T cells, all other evaluated anti-tumoral functions were virtually unchanged between TTFields and standard PBMC cultures (FIG. 2B). Note that TTFields did not fully abolish T cell proliferation, in agreement with the findings in FIG. 1.

An important feature of clinically effective T cells that are responsive to pathogens or to tumors is the ability of individual activated T cells to respond by means of several concurrent functions, also known as polyfunctionality. A co-expression analysis of T cell function performed on the dataset from FIG. 2B (FIG. 2C) showed that with the exception of proliferation, activated CD4+ and CD8+ T cells exhibited similar polyfunctional patterns under TTFields or standard culture conditions. The data illustrate shifts of T cells from parallel polyfunctional groups that differed only in proliferation. These findings also demonstrate that proliferation is independent of other key anti-tumoral T cell functions, as previously shown by others. Note that although CD107a is not a standard T helper marker, activated CD4+ T cells were shown to upregulate CD107a and secrete granzyme B 32. CD4+CD107+ T cells were reported to exhibit enhanced survival compared to their CD4+CD107a- counterparts.

The collected multiparametric data also enabled absolute enumeration of live cells according to cell type (CD4/8) and to proliferative status. As demonstrated in FIG. 1, the number of live T cells found in unstimulated samples cultured under TTFields was 85% that of controls (non-significant, p=0.48) (FIG. 2D-I). As in FIG. 1B, the PHA-stimulated cultures contained significantly fewer T cells (p=0.024) when incubated under TTFields compared to incubation under standard conditions (FIG. 2D-II). Importantly, there was no significant difference in the number of cells in these cultures when counting only the T cells which exhibited one or more activation parameters other than proliferation (FIG. 2D-III+IV, CD4+ p=0.85, CD8+ p=0.37). These results indicate that TTFields neither reduced the viability/number nor the functional responses in activated T cells that did not attempt to proliferate. Note that the group of activated non-proliferating cells that was evaluated here is a subgroup of stimulated, non-proliferating cells evaluated in FIG. 1B.

### iii. Key Anti-Tumoral Functions in GBM TILs are Unaffected by TTFields

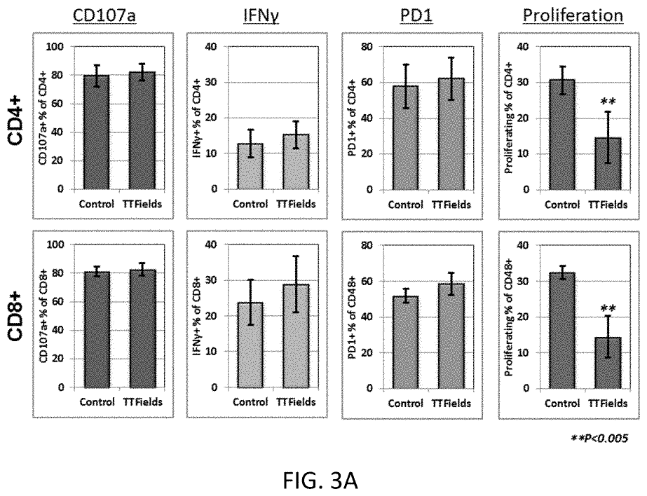

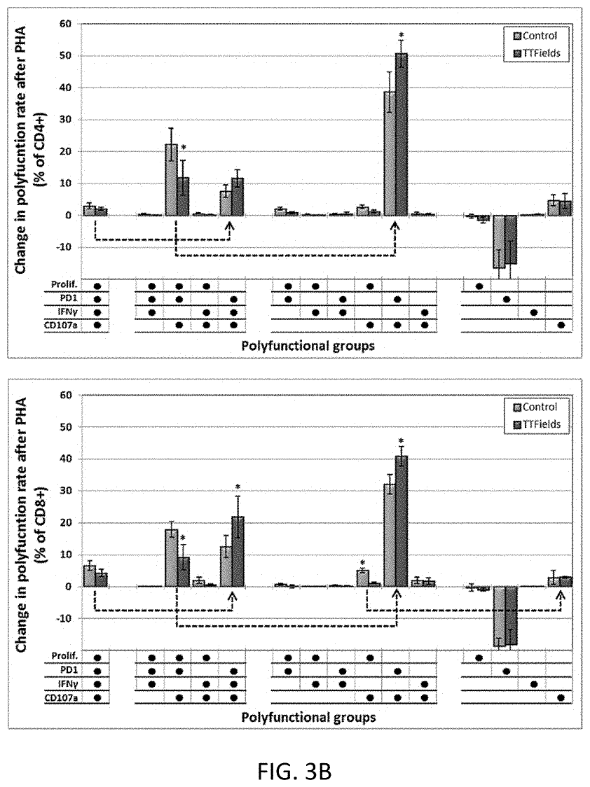

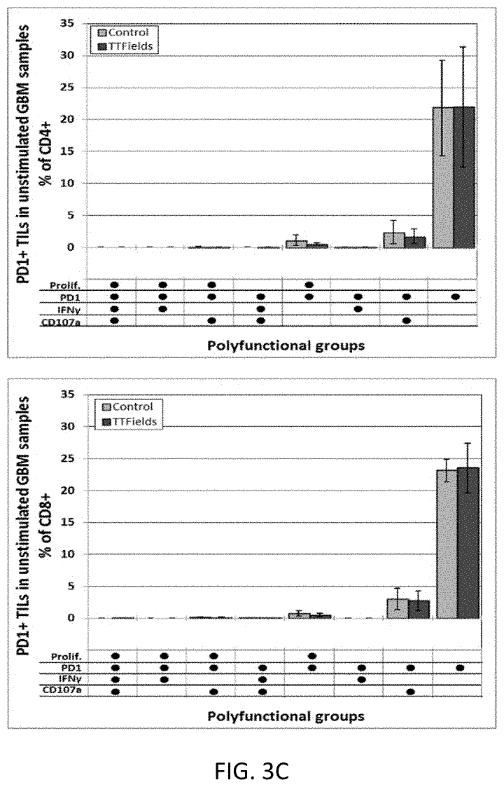

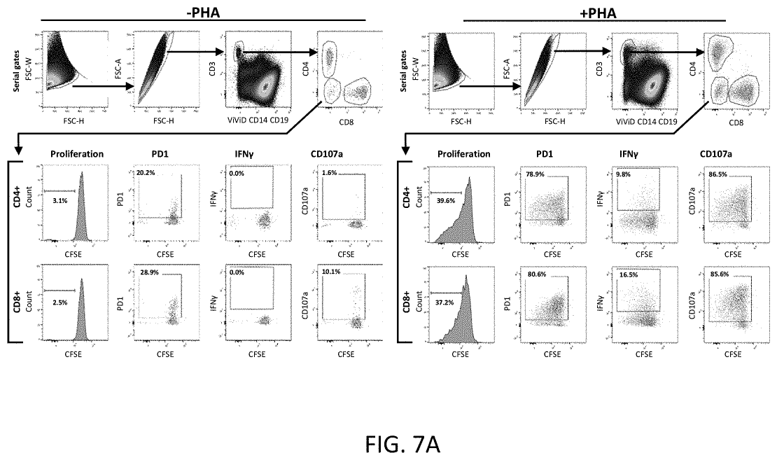

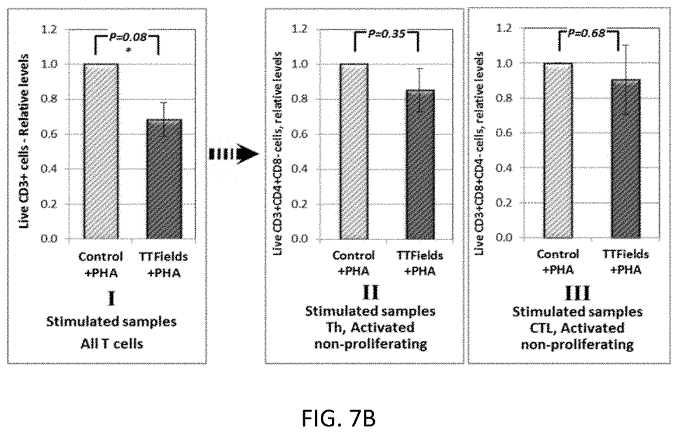

When compared to blood-borne T cells, TILs frequently distribute differently between memory/effector subsets and exhibit different effector and proliferation capacities. The GBM microenvironment is known to modify T cell phenotypes, and TILs may therefore respond differently than PBMC to TTFields. To examine the function of TILs under TTFields conditions, viably dissociated human GBM samples were incubated under standard or TTFields conditions and subjected to FACS analysis (gating detailed in FIG. 7A). As with PBMCs, TIL proliferation was inhibited but not fully abolished under TTFields, while all other evaluated T cell functions were unaffected (FIG. 3A). In addition, upon PHA stimulation, the TILs (both CD4+ and CD8+) exhibited comparable polyfunctional responses under TTFields and standard culture conditions (FIG. 3B).

Due to the considerable heterogeneity in TIL frequencies among the different patient-derived GBM samples used, consistent absolute cell enumeration (as in FIG. 2D) was not applicable. Therefore, a more limited analysis of the relative fractions of live TILs was performed (FIG. 7B). That analysis revealed no significant differences between control and TTFields-cultured GBM samples in the fraction of TILs responding to PHA stimulation by one or more functions other than proliferation (mirroring the findings in FIG. 2D).

### iv. Tumor Antigen-Specific T Cells (TASTs) Exposed to TTFields Exhibit Unaltered Viability and Function

TTFields are not expected to negatively affect TAST proliferation in tumor-draining lymph nodes found outside the treated field. However, TASTs may also proliferate within tumors where TTFields may have an effect. To date, there are no methods to clearly identify which TILs are also TASTs. However, it has been independently shown that the overwhelming majority of TASTs expresses PD1 on their surface. This characterization was relied on to evaluate the effect of TTFields on intratumoral TASTs by analyzing PD1+ TILs in unstimulated GBM cultures incubated under standard or TTFields conditions. Note that the rate of PD1+ T cells within GBM TILs was considerably higher than the rate of PD1+ T cells in the matching patients' PBMC (25% versus 3% in CD8, and 27% versus 7% in CD4 T cells, respectively, FIG. 7A). This reflects local activation and/or exhaustion of some TILs. Importantly, it was found that PD1+ TASTs under TTFields or standard culture conditions were functionally/polyfunctionally similar (FIG. 3C).

### v. Cytotoxic Capacity of CAR T Cells is Unaffected by TTFields

T cell-mediated cytotoxicity depends on the formation of a functional cytotoxic synapse. This requires extensive microtubule and centrosome realignment, which may plausibly be disrupted by TTFields. These findings, detailed above, revealed that cytotoxic degranulation (CD107a) is unhindered by TTFields. However, given that T cell cytotoxicity is essential for effective immunotherapy and that CD107a degranulation marker is a surrogate marker for tumor cell killing, it was necessary to directly assess cytotoxicity under TTFields conditions. This was accomplished by utilizing a CAR-T cell-based assay to directly evaluate cytotoxicity in a robust manner. CAR-T cells rely on the same intracellular machinery, and cytotoxicity is mediated by identical processes as those in non-engineered T cells, thus supporting the choice of assay. With CAR-T currently under clinical evaluation for GBM and many other solid tumors, this assay also evaluated the possible compatibility of CAR-T with TTFields treatment.

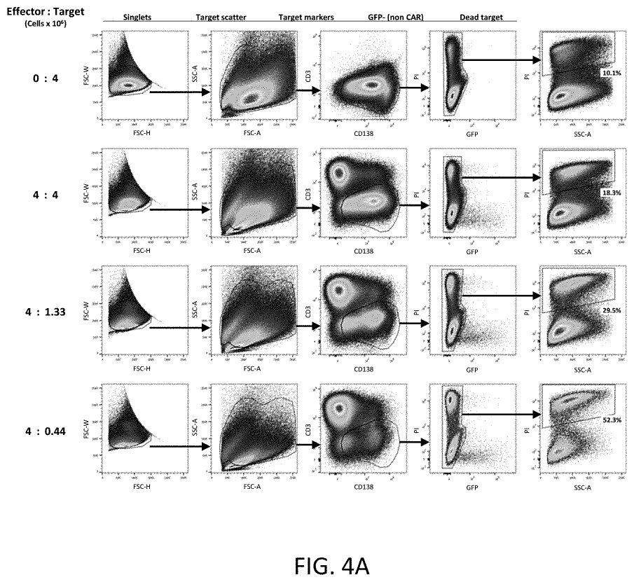

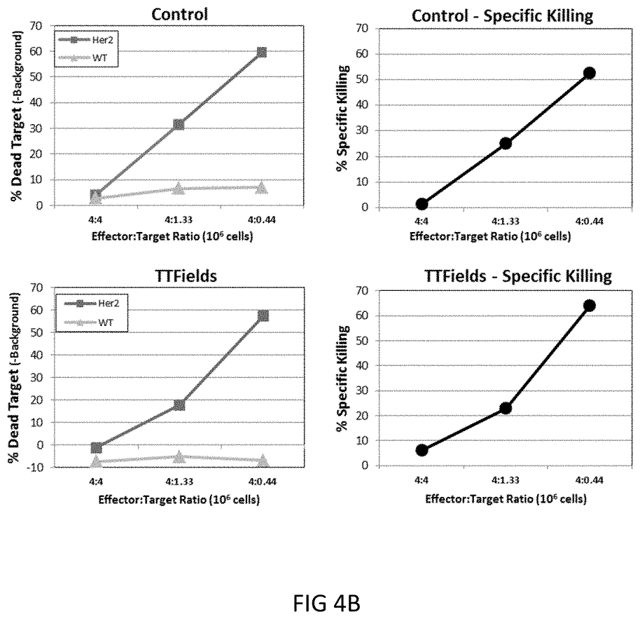

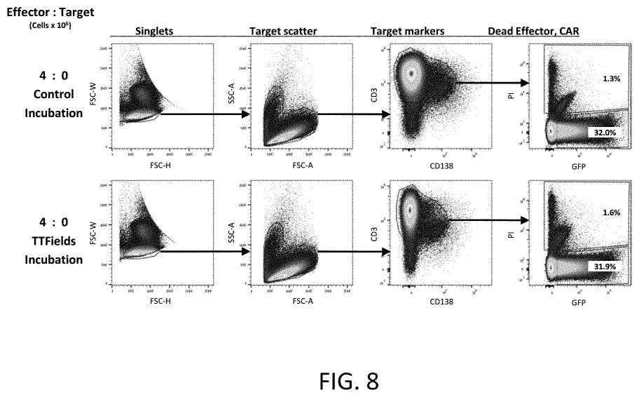

Human anti-HER2 CAR T cells were co-cultured with target cells, consisting of either parental (unaltered) CAG human myeloma cell line or CAG that ectopically expresses HER2 (FIG. 4A). Killing of CAG-HER2 by HER2-specific CAR-T cells was unaffected by TTFields (FIG. 4B). Alternative gating of the same dataset examining the effector cells confirmed that TTFields had no effect on the viability of CAR-T effectors during the course of the assay (FIG. 8). Taken together, these results demonstrate that TTFields do not interfere with T cell-mediated cytotoxicity.

### vi. Evaluating the Clinical Compatibility of TTFields with Anti-Tumoral Immunity

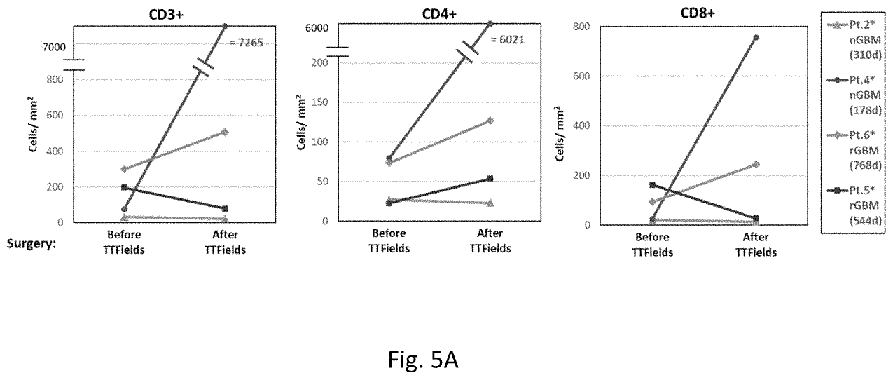

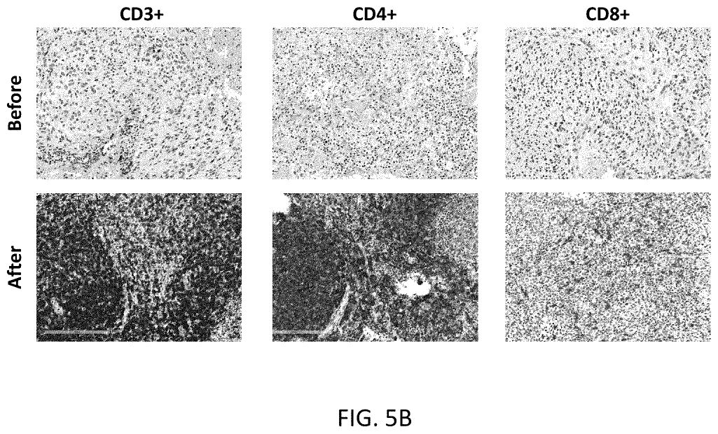

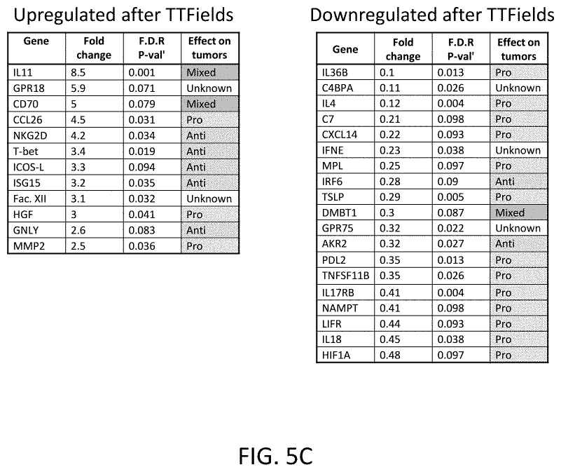

To support the in-vitro findings, the effect of TTFields on the immune response in clinical settings was examined. First, T cell infiltration rates were compared in GBM samples resected before and after TTFields treatment from four patients by performing immunohistochemical (IHC) staining for CD3, CD4 or CD8 (FIG. 5A, patient details in FIG. 12). While one patient's CD3+ and CD8+ TIL counts were slightly reduced, the infiltration rates in the other monitored patients were either unchanged or strongly increased (two patients) following TTFields treatment. The representative images from one patient in FIG. 5B demonstrate that TTFields therapy did not preclude dramatic increases in TIL density. T cells are not lost under in-vivo TTFields application and that TIL numbers can increase significantly in some cases.

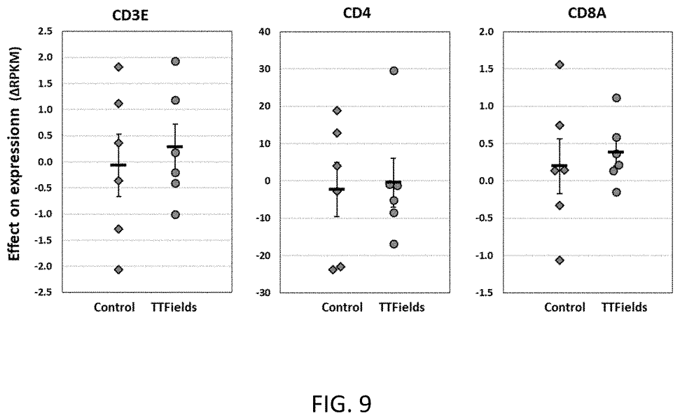

How TTFields modulate immune activity in GBM tissues were evaluated by studying their effects on gene expression. GBM samples were obtained before and following treatment from six patients treated by standard chemoradiation protocol (controls) and from six patients treated by chemoradiation and TTFields (FIG. 12). RNA sequencing and differential gene expression analyses of the samples demonstrated similar T cell infiltration trends as in the IHC (FIG. 9). Specifically, the net post-to-pre-treatment expression levels of the CD3, CD4 and CD8 genes (representing T cell infiltration) were slightly higher in the TTFields group than in the control group, both per individual patients and per the averaged group. While transcriptomic signature analysis is not as definitive as IHC, it nevertheless supports the IHC with additional samples evaluated and is in line with the IHC findings.

The effect of TTFields on a broad set of 712 immune-related genes was then evaluated using the above sequencing data. The table in FIG. 5C summarizes all genes within this comprehensive list found to be significantly up- or downregulated in the TTFields group compared to the control group. Genes with significantly altered expression were classified as pro- or antitumoral based on published literature, with some genes classified as having mixed or unknown roles (FIG. 13). Of the 12 genes found to be upregulated in TTFields vs controls, 5 were anti-tumoral while 3 were pro-tumoral. The anti-tumoral upregulated genes serve key roles in T cell and NK cell anti-tumoral responses, including the hallmark Th1 transcription factor t-bet, NK activatory receptor NKG2D, co-stimulatory ICOS-L and the cytotoxic granulysin (GNLY). TTFields upregulated pro-tumoral genes (HGF, MMP2, CCL26) seem to be more related to tumor progression than to specific immune functions.

Of the 20 genes that were downregulated in TTFields versus controls, 13 were pro-tumoral while 2 were anti-tumoral. TTFields' downregulated anti-tumoral genes were IRF6 (shown to exhibit antitumoral effects in non-brain tumors) and ACKR2 (a chemokine scavenger receptor). The pro-tumoral genes downregulated in the TTFields group relative to controls included pro-inflammatory genes (IL36B, IL18, C7), genes that reduce T cell infiltration and promote infiltration of immune suppressive cells (CXCL14), T cell exhaustion-inducing ligand (PDL2), and genes that polarize T helpers to ineffective subtypes (IL4, IL17RB) and the pro-angiogenic factor, HIF1α. Overall, the data demonstrated a significant shift in immune-related gene expression from pro-tumoral to anti-tumoral genes following TTFields+chemoradiation treatment when compared to treatment by chemoradiation alone (p=0.026 Fisher exact).

Results presented thus far raise the prospect that TTFields-treated GBM cells can be harnessed to induce adaptive immunity against GBM tumors. To test this concept, KR158-luc cells were treated with TTFields for 72 hrs. first before stereotactically implanting them into the posterior right frontal cerebrum of C57BL/6J mice to provide both immunogens and adjuvant signals, while avoiding confounding effects of TTFields on tumor stromal and immune cells. Importantly, it was confirmed that STING and AIM2-dependent upregulation of PICs, T1IFNs and T1IRGs in KR158-luc cells persisted for at least 3 days after TTFields cessation, providing the rationale for their use as an immunizing vehicle. Vaccinated animals were immunophenotyped and their brains examined histologically 2 weeks after implantation or monitored for tumor growth by bioluminescence imaging (BLI) and overall survival (OS). To test for an anti-tumor memory response, surviving animals were re-challenged at day 100 and the same number of vaccine-naïve, sex-matched, 6-8 weeks old C57BL/6J controls with a 2-fold higher number of non-treated KR158-luc cells and compared immune responses and OS of the 2 groups.

At day 7 (D7) post implantation, all groups developed comparable BLI signals, confirming that primary tumor establishment was equivalent in all conditions. Subsequently, however, all but 1 animal (38 of 39 or 97%) in the 3 control groups, i.e., scrambled shRNA/non-treated (Sc), STING-AIM2 DKD/TTFields-treated (DKD-TTF), and STING-AIM2 DKD/non-treated (DKD) developed progressive brain tumors and succumbed by day 100 with median OS (mOS) of 45 days. In contrast, 10 of 15 (66%) animals receiving scrambled shRNA/TTFields-treated cells (Sc-TTF) had no detectable tumor at day 100 with mOS not reached. When these 10 surviving Sc-TTF animals were re-challenged with 2-fold parental KR158-luc cells, 6 (60%) survived for at least 144 more days without any detectable tumor, as compared to none of the 12 naïve controls surviving past 45 days with mOS of only 38 days despite their being much younger in age. The 4 Sc-TTF mice that succumbed by 100 days still exhibited a significant delay in tumor growth and improved survival compared to the naïve controls. In summary, 40% (6 of 15) animals immunized with Sc-TTF cells developed robust anti-tumor immunity and another 25% (4 of 15) derived partial immunity in a TTFields, STING and AIM2-dependent manner—a remarkable feat for KR158, a poorly immunogenic model that closely resembles human GBM.

To define the immunological basis of these positive clinical observations, we harvested the ipsilateral deep cervical lymph nodes (dcLNs), thought to directly drain the brain and the ipsilateral head and neck, for immunophenotyping. Compared to animals receiving Sc cells, the fraction of DCs in dcLNs increased in mice immunized with Sc-TTF cells, which was reversed when DKD-TTF cells were injected. DKD cells resulted in no difference in DCs in dcLNs compared to Sc cells, indicating that STING and AIM2 only became dominant with TTFields. Importantly, of the DCs in dcLNs, the fraction of activated DCs (CD80/CD86⁺) doubled when Sc-TTF cells were implanted instead of Sc, DKD-TTF or DKD cells, which coincided with an increase in the fractions of early activated CD69⁺ CD4 and CD8 T cells, even though the total and activated CD4 and CD8 fractions had not increased yet by this time.

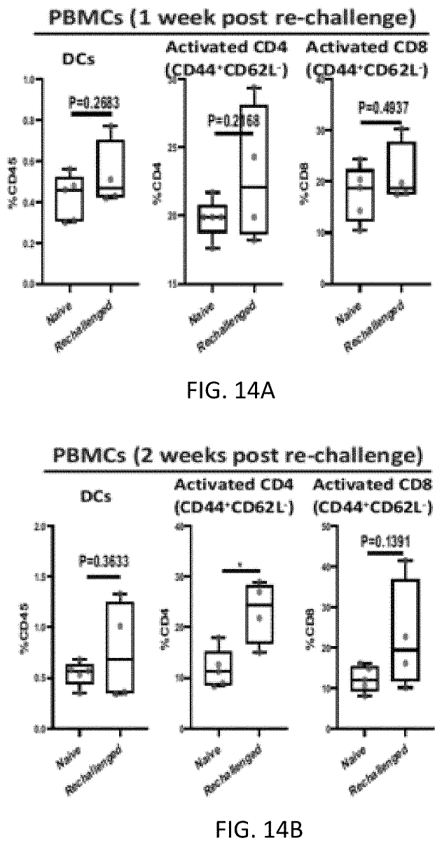

Next, the peripheral immune compartment was examined for the emergence of a memory adaptive response to KR158 tumors by temporally immunophenotyping splenocytes and peripheral blood mononuclear cells (PBMCs) at Week 2 post primary immunization and then at Week 1 and 2 post re-challenge, with minimal changes expected at the earlier time point. At Week 2 post immunization, as predicted, there was only a trend of increase in DCs and no change in lymphocytes in PBMCs except that CD69⁺ CD8 T cells was higher in Sc-TTF animals. Surprisingly, however, we detected an increase in total and activated DCs and CD69⁺ CD8 T cells in splenocytes from Sc-TTF animals, compared to controls at this time, attesting to the strength of TTFields-induced immune stimulation. Indeed, we found an increase in infiltration of T (CD3) and CD8 T (CD3⁺CD8⁺) cells in Sc-TTF tumors compared to other control tumors. Upon re-challenge, the fractions of DCs and activated CD4 and CD8 T cells rapidly increased at Week 1 and rose further at Week 2 (FIG. 14a-b), while those of CD69⁺ CD4 and CD8 T cells increased only at Week 1, not Week 2, in the rechallenged Sc-TTF cohort as compared to the vaccine naïve controls. Of note, no differences in myeloid derived suppressive cells (CD11b⁺/Ly6g/Ly6c⁺) and macrophages were detected in the different cohorts at any time. To confirm the presence of central memory (CM) T cells in the 6 long-term surviving rechallenged Sc-TTF mice, we measured the fractions of CM (CD44⁺CD62L⁺) CD4 and CD8 T cells in their dcLNs and spleens at 20 weeks post rechallenge. For control, we implanted the same number of KR158-luc cells into an age- and sex-matched cohort of 6 naïve mice and analyzed their dcLNs and spleens 2 weeks later. The fractions of CM and effector (CD44⁺CD62L⁻) T cells were consistently higher in Sc-TTF mice than in the naïve controls (FIG. 14c).

### vii. Adaptive Immune Activation by TTFields in GBM Patients via a T1IRG-Based Trajectory

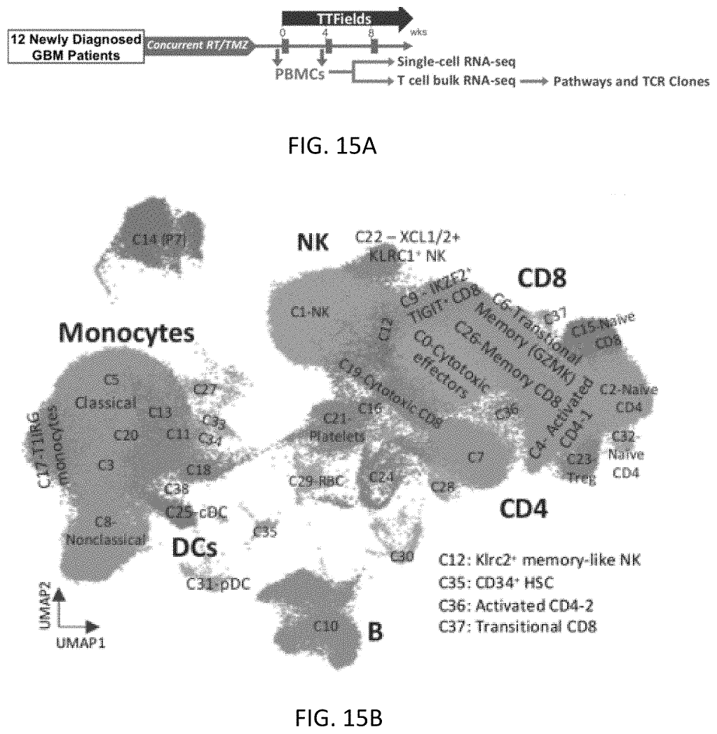

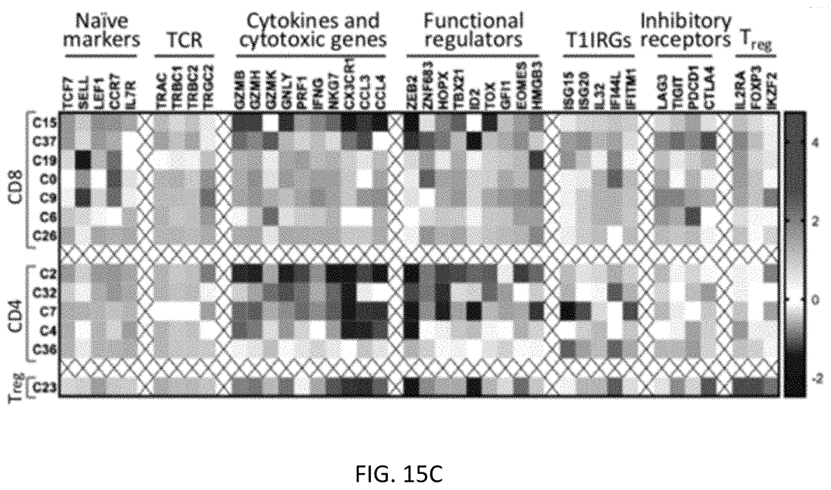

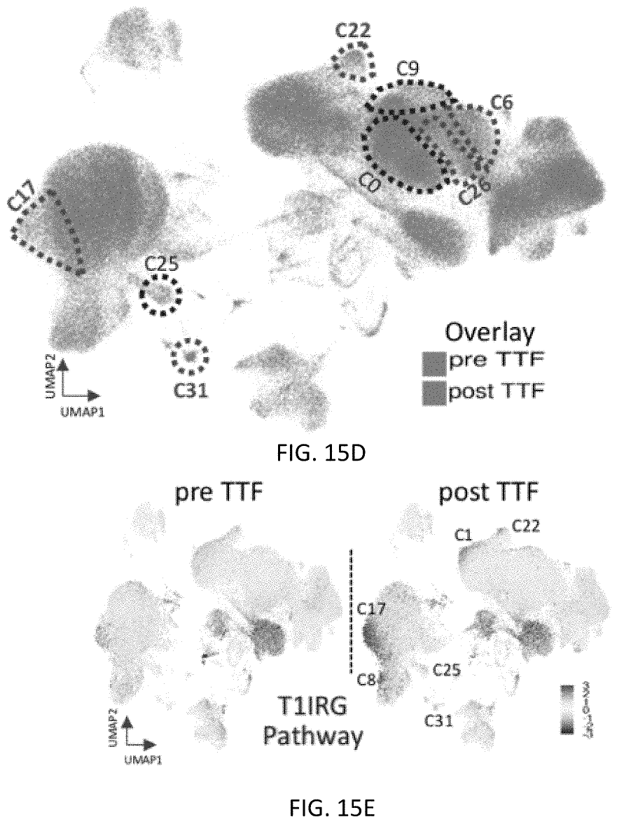

TTFields similarly activate adaptive immunity in patients with GBM, specifically through a T1IRG-based trajectory, and that a gene signature linking TTFields to adaptive immunity is identifiable. To that end, PBMCs were collected from 12 adult patients with newly diagnosed GBM after completing chemoradiation at the following 2 times—within 2 weeks before and about 4 weeks after initiation of TTFields and TMZ (FIG. 15a)—to perform 1) single-cell RNA-seq (scRNA-seq) to identify the cell types and subtypes responsible for TTFields effects; and 2) deep bulk RNA-seq of isolated T cells to identify a gene signature that captures broad effects of TTFields-induced T1IFNs across T cell subtypes. The high sequencing depth also enabled a focused clonal analysis of the most abundant T cell receptor (TCR) clones to provide direct evidence of adaptive immune activation by TTFields.

In total, 193,760 PBMCs were resolved in the 24 paired samples. Using the graph-based cell clustering technique UMAP55, we partitioned the graph using increasing resolution parameter values (0.1, 0.3, 1, 3, 5 and 10). Resolution 1 was chosen as it produced reasonably sized clusters, partitioning PBMCs into 38 biologically recognized subtypes of 8 main cell types (FIG. 15b). Cluster 14 (C14), contributed entirely by Patient 7 (P7) alone, contained an altered monocyte population of unclear significance. To more accurately annotate T cell clusters, we assembled a gene set containing cell type markers and functional regulators, gleaned from the UMAP clustering and literature review 56-59 (FIG. 15c). For instance, C15 contained naïve CD8 T cells, while C37 expressing granzyme K (GZMK) constituted transitional or partially activated CD8 T cells. Cytotoxic effectors populated C0 and differed from exhausted effectors of C9 in that C0 expressed the cytotoxic regulator ZNF68361,62 and lacked the inhibitory marker TIGIT and the regulatory T cell (Treg) factor IKZF263 found in C9 (FIG. 15c). C6 and C26 comprised transitional and long-lived memory CD8 T cells, respectively, and distinguished from each other by GZMK (C6), GZMB64, CCL365 and CCR7 (C26) (FIG. 15c).

An overlay of the pre- and post-TTFields UMAP graphs revealed proportional increases in several clusters (FIG. 15d). Consistent with TTFields inducing the immune system via a T1IFN-based trajectory, we found higher proportions of plasmacytoid DCs (pDCs) (C31) (FIG. 15f), a specialized DC subtype that is both a direct target and the highest producer among DC subtypes of T1IFNs and key in linking the innate and adaptive immune systems 68-70, and of a monocyte subtype (C17) expressing T1IRGs including IFI44L, MX1 and ISG15 (FIG. 15g). There was also a trend of increase in the XCL1/2+KLRC1+ subtype (C22) of NK cells, another major T1IFN-responsive innate immune cell type (FIG. 15h). To confirm that these 3 clusters constituted the front of the TTFields-induced T1IRG-based pathway trajectory, a global survey was conducted at the single cell level before and after TTFields for the mean expression of GO-0034340, a major T1IRG pathway with 99 genes annotated by Gene Ontology. Indeed, this T1IRG pathway formed an upregulated arc in response to TTFields that spanned these very 3 clusters and extended to other innate cell types, including non-classical monocytes (C8), classical NK cells (C1) and classical DCs or cDCs (C25) (FIG. 15e). When gene coverage was expanded to all genes and pathways or cell-specific pathways using the gene set enrichment analysis (GSEA74), there was widespread expression upregulation in pDCs in all 9 patients with detectable pre- and post-TTFields pDCs, specifically in T1IRG and DC-regulatory pathways (FIGS. 15m-n). Moreover, post-TTFields pDCs upregulated the IFNγ (T2IFN) pathway known to promote DC maturation. Although no numerical increase was observed, just as in the KR158 model where the increase was noted mostly in dcLNs, cDCs in 11 PBMCs exhibited pervasive post-TTFields upregulation of genes and pathways analogous to those in pDCs (FIGS. 15o-p). Likewise, TTFields treatment led to global upregulation in C17 and C22 and in other innate clusters, albeit with higher inter-patient variations. Taken together, these results confirmed robust gene upregulation in DCs and innate cells post TTFields in GBM patients, specifically following a T1IRG-based trajectory.

Whether effector T cells were activated following TTFields-induced DC activation, as observed in the KR158 model, was examined. Although cytotoxic (C0) and exhausted (C9) effectors did not increase in proportion, their expression profiles and that of activated CD4 (C4) showed global gene upregulation post TTFields to varying degrees across patients and clusters (FIGS. 15i-j). GSEA of C0 revealed enrichment in MHC-binding, NFκB, IL-1, and Toll-like-receptor-3 pathways among others, which have been specifically implicated in antigen-specific CD8 effector activation and expansion. Of note, C0 cells also upregulated the Fas/FasL pathway, known to promote activation-induced cell death in cytotoxic effectors, presumably contributing to the lack of increase in C0 as they transition to memory T cells at 4 weeks after TTFields start. Consistent with this notion, a trend was detected of increase in long-lived memory CD8 T cells (C26), which coincided with a contraction in transitional memory CD8 T cells (C6) (FIG. 15k-l), with both exhibiting global upregulation across patients to varying degrees. GSEA of C26 and C6 showed enrichment in shared regulatory pathways previously implicated in memory T cells development and maintenance, including the mTOR and complement activation pathways.

### 3. Discussion

Knowledge of the effect of TTFields on T cells is critical when considering the pivotal role of T cells in anti-tumoral immune responses. By evaluating well-established key markers of antitumoral T cell function, these findings (FIG. 1-3) indicate that TTFields operated at therapy-relevant conditions do not hinder any of these functions aside from proliferation. TTFields has low or no effect on unstimulated T cells, and its effects on stimulated T cells are minor so long as they do not attempt to proliferate. This is also supported by a polyfunctional analysis (FIG. 2D) which showed that the amount of T cells that responded to stimulation by any function other than proliferation was similar in TTFields and standardly cultured samples. These findings hold not only to cells derived from healthy donor blood, but also when analyzing viably dissociated GBM tumor samples and examining infiltrating T-cell functionality. Applying a CAR-based cytotoxicity assay, it was demonstrated that the key anti-tumoral function of CTLs is not impeded by TTFields and that CAR-T cells demonstrate apparent compatibility with TTFields technology. This is the first time CAR T functionality was examined in the context of TTFields.

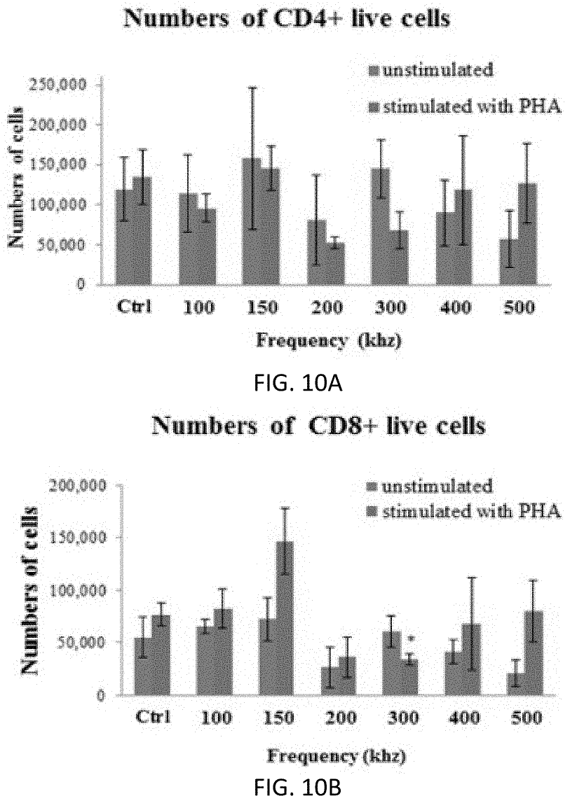

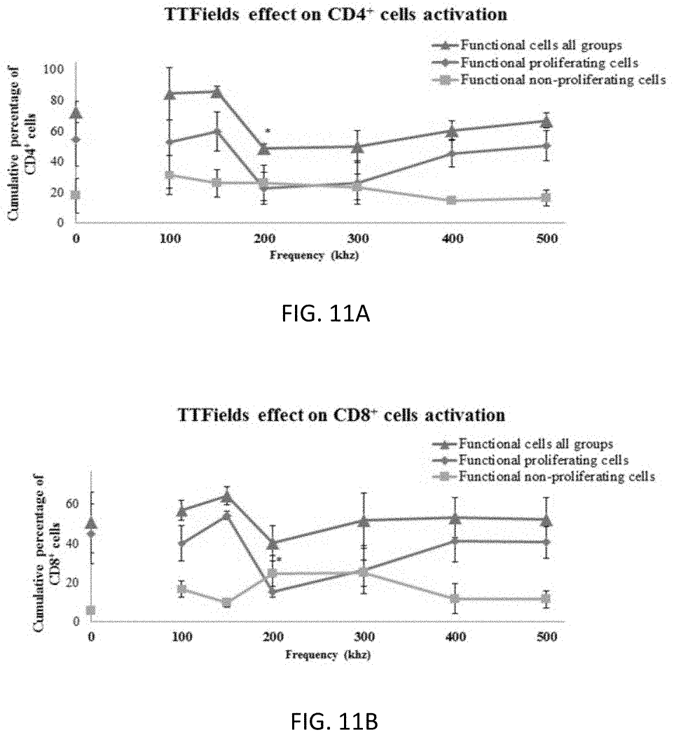

An in-depth study of T cell functionality under the entire clinically relevant frequency spectrum of TTFields is not within the scope of this study. Notwithstanding, since the recent approval of TTFields for mesothelioma employs a field frequency of 150 kHz rather than 200 kHz (as in GBM treatment), it was further tested whether the phenomenon observed in 200 kHz applies to additional frequencies. The evaluation of 100, 150, 200, 300, 400 and 500 kHz revealed that 300 kHz and, even more potently, 200 kHz to be the frequencies with the strongest negative effect on proliferating T cells. Frequencies such as 100 kHz and 150 kHz showed no negative effects on the number of viable T cell and no effect on the T cells functionality (FIGS. 10 and 11). FIG. 10 shows no significant changes in the numbers of unstimulated T cells (blue bars) between treatments. In contrast, some reduction was found in total counts of stimulated CD4 (A) or CD8 (B) T cell, especially in the 200 and 300 kHz range. This reiterates data in FIG. 1B showing the TTFields have minimal effect on the viability of unstimulated T cells in 200 kHz. FIG. 11 demonstrates significant decreases in the numbers of proliferating, function positive, T cells at 200 kHz. The effect was observed both on CD4+ (P<0.05) and on CD8+ T cells (NS), and was also observed in 300 kHz frequency (NS) (circle). The decrease in the fraction of the functional and proliferating T cells co-occurred with an increase in functional T cells which did not attempt to proliferate (square). This phenomenon was more apparent in the CD8+ T cells in 200 kHz (P<0.05) and 300 kHz (NS). The shift from functional proliferating groups to functional non-proliferating groups was mainly found in populations that differed from each other in one function—proliferation, as in FIG. 3B (TTFields frequency—200 kHz).

A differential response of tumor cell lines to various frequencies was previously demonstrated by others, and matching TTFields frequency to each treated cancer indication is a cornerstone of this technology. Appropriate consideration of TTFields parameters, such as field frequency, intensity and administration protocols, should balance between effective tumor cell inhibition while minimizing the disruption of T cell function.

The potential inhibitory impact of TTFields on intratumoral T cell proliferation may be of some concern. It is, however, not yet clear if and to what extent intra-tumoral TASTs proliferate within GBM and other solid malignancies. Expanded TCRs clonotypes found among TIL populations in GBM and other tumors indicate that proliferation of TASTs occurs but not of where it had occurred. Murine studies have yielded conflicting findings with respect to intra-brain T cell proliferation, with some having shown that brain antigen-specific activated T cells do not proliferate within the brain, and others demonstrating that the brain microenvironment may support in-situ proliferation under some circumstances. Brain- or tumor-specific T cell priming and proliferation were shown to occur in the meningeal lymphatics and within the deep cervical lymph nodes found outside the TTFields-affected field.

Importantly, several studies have indicated that only peripheral T cells, and not intratumoral ones, bear sustained proliferative capabilities, and that T cell activation and proliferation must occur within peripheral lymphoid organs in order to achieve tumor eradication. A recent study provided evidence of clonotypic expansion of effector-like T cells in 4 different tumor types, both within the tumor as well as in adjacent normal tissue and within peripheral blood. Patients exhibiting this wide clonotypic expansion pattern were those most likely to respond to anti-PDL1 therapy. The authors suggested that intratumoral T cells (especially in anti-PDL1-responsive patients) are replenished by non-exhausted tumor-specific T cells arriving from outside the tumor. If this also applies to brain tumors, it is likely that TTFields-induced reduction of activated proliferating T cells will have little clinically meaningful effect. This should also hold true for any tumor which may elicit an extra-tumoral TAST expansion.

Although this work is primarily at cell-level and mechanistic, IHC and RNA-Seq analyses were performed on tissue samples from TTFields-treated patients to support and extend the findings to the clinical setting. The RNA-seq analysis is the first transcriptomic analysis of the effects of TTFields by using matched, patient-derived samples. The IHC results obtained from 4 TTFields-treated patients were complemented by separate RNA-Seq data obtained from 6 TTFields- and 6 standard-treated patients, and both showed that TTFields neither precluded nor reduced the accumulation of T cells within GBM.

The transcriptional analyses of pre-and-post treatment GBM tissue samples, which compared standard chemoradiation to chemoradiation+TTFields, provided further support for the IHC data. However, their main importance is as an indication of a shift in the transcriptional profile of intra-tumoral immune cells from a Th2/Th17 one that is associated with GBM patients who have an unfavorable prognosis to a cellular/Th1-oriented profile that is frequently associated with a more positive prognosis.

The in-vitro/ex-vivo mechanistic part of this study focused upon evaluating the potential deleterious effects of TTFields on T cells, while the clinical part demonstrated several immune-related beneficial effects of TTFields therapy on the systemic level, namely, a significant shift from pro-tumoral to anti-tumoral gene expression. The potential of TTFields to operate alongside and, in some cases, to synergize with antitumoral responses had been demonstrated when TTFields therapy was shown to enhance T cell infiltration in various tumor models. A recent study provided direct evidence of TTFields inducing ICD in multiple mouse tumor cell lines. It was demonstrated that treatment with a PD1 inhibitor and TTFields provided mice with better anti-tumor protection than either treatment alone. In one of the two murine tumor models tested, T cells also increased both in numbers and in IFNγ production when TTFields treatment was combined with PD1. Clinical data in GBM patients had demonstrated a correlation overall survival benefit of TTFields with higher blood T-cell counts and with low (<4.1 mg) or no administration of immunosuppressive dexamethasone. While these human data do not revel cause and effect, they do correlate with the mouse in-vivo and in-vitro data, further substantiating the contribution of an intact immune compartment to the clinical benefit of TTFields.

Given these findings and the nature of TTFields, how should oncologists approach combining this modality with immunotherapy? CPIs represent one promising possibility. A triple combination of PD1 and CTLA4 inhibitors with an immune-stimulating oncolytic virus has been described. This combination enhanced T cell infiltration into gliomas and provided better survival than any of those agents alone or as doublets. TTFields can take the place of the oncolytic ICD-inducing virus, or use of radiotherapy inducing similar immune consequences, turning the tumor to an in-situ vaccine.

The first studies on combining TTFields with various immunotherapeutics are already underway. The human ex-vivo data together with the supporting clinical findings indicate a sustained capacity of T cells to survive and function under TTFields. These findings provide support for the rationale for those clinical studies and provide the knowledge needed to interpret the results of these and of future combination studies involving TTFields and immunotherapy.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Kirson, E. D., Z. Gurvich, R. Schneiderman, E. Dekel, A. Itzhaki, Y. Wasserman, et al., Disruption of cancer cell replication by alternating electric fields. Cancer Res, 2004. 64(9): 3288-95.
2. Wang, Y., M. Pandey, and M. T. Ballo, Integration of Tumor-Treating Fields into the Multidisciplinary Management of Patients with Solid Malignancies. Oncologist, 2019. 24(12).
3. Mittal, S., N. V. Klinger, S. K. Michelhaugh, G. R. Barger, S. C. Pannullo, and C. Juhasz, Alternating electric tumor treating fields for treatment of glioblastoma: rationale, preclinical, and clinical studies. J Neurosurg, 2017: 1-8.
4. Ceresoli, G. L., J. G. Aerts, R. Dziadziuszko, R. Ramlau, S. Cedres, J. P. van Meerbeeck, et al., Tumour Treating Fields in combination with pemetrexed and cisplatin or carboplatin as first-line treatment for unresectable malignant pleural mesothelioma (STELLAR): a multicentre, single-arm phase 2 trial. The Lancet Oncology, 2019. 20(12): 1702-1709.
5. Organization, W. H., World Cancer Report 2014. World Cancer Report, ed. W. C. Stewart BW. Vol. 3. 2014: IARC publications.
6. Sampson, J. H., M. V. Maus, and C. H. June, Immunotherapy for Brain Tumors. J Clin Oncol, 2017: JCO2017728089.
7. Gibney, G. T., L. M. Weiner, and M. B. Atkins, Predictive biomarkers for checkpoint inhibitor-based immunotherapy. Lancet Oncol, 2016. 17(12): e542-e551.
8. Melero, I., D. M. Berman, M. A. Aznar, A. J. Korman, J. L. Perez Gracia, and J. Haanen, Evolving synergistic combinations of targeted immunotherapies to combat cancer. Nat Rev Cancer, 2015. 15(8): 457-72.
9. Lim, M., Y. Xia, C. Bettegowda, and M. Weller, Current state of immunotherapy for glioblastoma. Nature reviews Clinical oncology, 2018. 15(7): 422.
10. Sharma, P. and J. P. Allison, The future of immune checkpoint therapy. Science, 2015. 348(6230): 56-61.
11. Hu, Z. I., H. L. McArthur, and A. Y. Ho, The Abscopal Effect of Radiation Therapy: What Is It and How Can We Use It in Breast Cancer? Curr Breast Cancer Rep, 2017. 9(1): 45-51.
12. Kroemer, G., L. Galluzzi, O. Kepp, and L. Zitvogel, Immunogenic cell death in cancer therapy. Annu Rev Immunol, 2013. 31: 51-72.
13. Postow, M. A., M. K. Callahan, C. A. Barker, Y. Yamada, J. Yuan, S. Kitano, et al., Immunologic correlates of the abscopal effect in a patient with melanoma. N Engl J Med, 2012. 366(10): 925-31
14. Giladi, M., T. Voloshin, A. Shteingauz, M. Munster, R. Blat, Y. Porat, et al., Alternating electric fields (TTFields) induce immunogenic cell death resulting in enhanced antitumor efficacy when combined with anti-PD-1 therapy. The Journal of Immunology, 2016. 196(1 Supplement): 75.26-75.26.
15. Voloshin, T., S. Davidi, Y. Porat, A. Shteingauz, M. Munster, N. Kaynan, et al., Immunomodulatory Effect of Tumor Treating Fields (TTFields) Results in Enhanced Antitumor Efficacy When Combined with Anti-PD-1 Therapy in Mouse Model of Lung Cancer. International Journal of Radiation Oncology⋅Biology⋅Physics, 2019. 104(1): 237.
16. Swanson, K. D., E. Lok, and E. T. Wong, An Overview of Alternating Electric Fields Therapy (NovoTTF Therapy) for the Treatment of Malignant Glioma. Curr Neurol Neurosci Rep, 2016. 16(1): 8.
17. Chen, D., N. Thomas, and D. D. Tran, TTFields induces immunogenic cell death and STING pathway activation through cytoplasmic double-stranded DNA in glioblastoma cells. 2019, AACR.
18. Kirson, E. D., M. Giladi, Z. Gurvich, A. Itzhaki, D. Mordechovich, R. S. Schneiderman, et al., Alternating electric fields (TTFields) inhibit metastatic spread of solid tumors to the lungs. Clin Exp Metastasis, 2009. 26(7): 633-40.
19. Voloshin, T., N. Kaynan, S. Davidi, Y. Porat, A. Shteingauz, R. S. Schneiderman, et al., Tumor-treating fields (TTFields) induce immunogenic cell death resulting in enhanced antitumor efficacy when combined with anti-PD-1 therapy. Cancer Immunology, Immunotherapy, 2020: 1-14.
20. Wong, E., E. Lok, S. Gautam, and K. Swanson, Dexamethasone exerts profound immunologic interference on treatment efficacy for recurrent glioblastoma. British journal of cancer, 2015. 113(2): 232.
21. Volovitz, I., N. Shapira, H. Ezer, A. Gafni, M. Lustgarten, T. Alter, et al., A non-aggressive, highly efficient, enzymatic method for dissociation of human brain-tumors and brain-tissues to viable single-cells. BMC Neurosci, 2016. 17(1): 30.
22. Globerson-Levin, A., T. Waks, and Z. Eshhar, Elimination of progressive mammary cancer by repeated administrations of chimeric antigen receptor-modified T cells. Mol Ther, 2014. 22(5): 1029-38.
23. Porat, Y., M. Giladi, R. S. Schneiderman, R. Blat, A. Shteingauz, E. Zeevi, et al., Determining the Optimal Inhibitory Frequency for Cancerous Cells Using Tumor Treating Fields (TTFields). J Vis Exp, 2017; (123).
24. Kirson, E. D., V. Dbaly, F. Tovarys, J. Vymazal, J. F. Soustiel, A. Itzhaki, et al., Alternating electric fields arrest cell proliferation in animal tumor models and human brain tumors. Proc Natl Acad Sci U S A, 2007. 104(24): 10152-7.
25. Lamoreaux, L., M. Roederer, and R. Koup, Intracellular cytokine optimization and standard operating procedure. Nat Protoc, 2006. 1(3): 1507-16.
26. Love, M. I., W. Huber, and S. Anders, Moderated estimation of fold change and dispersion for RNA-seq data with DESeq2. Genome biology, 2014. 15(12): 550.
27. Burkhardt, J. K., E. Carrizosa, and M. H. Shaffer, The actin cytoskeleton in T cell activation. Annu Rev Immunol, 2008. 26: 233-59.
28. Hottinger, A. F., P. Pacheco, and R. Stupp, Tumor treating fields: a novel treatment modality and its use in brain tumors. Neuro-oncology, 2016. 18(10): 1338-1349.
29. Betts, M. R., J. M. Brenchley, D. A. Price, S. C. De Rosa, D. C. Douek, M. Roederer, et al., Sensitive and viable identification of antigen-specific CD8+ T cells by a flow cytometric assay for degranulation. J Immunol Methods, 2003. 281(1-2): 65-78.
30. Betts, M. R., M. C. Nason, S. M. West, S. C. De Rosa, S. A. Migueles, J. Abraham, et al., HIV nonprogressors preferentially maintain highly functional HIV-specific CD8+ T cells. Blood, 2006. 107(12): 4781-9.
31. Restifo, N. P., M. E. Dudley, and S. A. Rosenberg, Adoptive immunotherapy for cancer: harnessing the T cell response. Nat Rev Immunol, 2012. 12(4): 269-81.
32. Lin, L., J. Couturier, X. Yu, M. A. Medina, C. A. Kozinetz, and D. E. Lewis, Granzyme B secretion by human memory CD4 T cells is less strictly regulated compared to memory CD8 T cells. BMC Immunol, 2014. 15: 36.
33. Terahara, K., H. Ishii, T. Nomura, N. Takahashi, A. Takeda, T. Shiino, et al., Vaccine-induced CD107a+ CD4+ T cells are resistant to depletion following AIDS virus infection. J Virol, 2014. 88(24): 14232-40.
34. Mahnke, Y. D., T. M. Brodie, F. Sallusto, M. Roederer, and E. Lugli, The who's who of T-cell differentiation: human memory T-cell subsets. Eur J Immunol, 2013. 43(11): 2797-809.
35. Spitzer, M. H., Y. Carmi, N. E. Reticker-Flynn, S. S. Kwek, D. Madhireddy, M. M. Martins, et al., Systemic Immunity Is Required for Effective Cancer Immunotherapy. Cell, 2017. 168(3): 487-502 e15.
36. Ahmadzadeh, M., L. A. Johnson, B. Heemskerk, J. R. Wunderlich, M. E. Dudley, D. E. White, et al., Tumor antigen-specific CD8 T cells infiltrating the tumor express high levels of PD-1 and are functionally impaired. Blood, 2009. 114(8): 1537-44.
37. Donia, M., J. W. Kjeldsen, R. Andersen, M. C. W. Westergaard, V. Bianchi, M. Legut, et al., PD-1(+) Polyfunctional T Cells Dominate the Periphery after Tumor-Infiltrating Lymphocyte Therapy for Cancer. Clin Cancer Res, 2017. 23(19): 5779-5788.
38. de la Roche, M., Y. Asano, and G. M. Griffiths, Origins of the cytolytic synapse. Nat Rev Immunol, 2016. 16(7): 421-32.
39. Ramos, C. A., H. E. Heslop, and M. K. Brenner, CAR-T Cell Therapy for Lymphoma. Annu Rev Med, 2016. 67: 165-83.
40. O'Rourke, D. M., M. P. Nasrallah, A. Desai, J. J. Melenhorst, K. Mansfield, J. J. D. Morrissette, et al., A single dose of peripherally infused EGFRvIII-directed CAR T cells mediates antigen loss and induces adaptive resistance in patients with recurrent glioblastoma. Sci Transl Med, 2017. 9(399).
41. Porat, Y., M. Giladi, R. S. Schneiderman, R. Blat, A. Shteingauz, E. Zeevi, et al., Determining the optimal inhibitory frequency for cancerous cells using tumor treating fields (TTFields). JoVE (Journal of Visualized Experiments), 2017; (123): e55820.
42. Sims, J. S., B. Grinshpun, Y. Feng, T. H. Ung, J. A. Neira, J. L. Samanamud, et al., Diversity and divergence of the glioma-infiltrating T-cell receptor repertoire. Proc Natl Acad Sci U S A, 2016. 113(25): E3529-37.
43. Aloisi, F., F. Ria, and L. Adorini, Regulation of T-cell responses by CNS antigen presenting cells: different roles for microglia and astrocytes. Immunol Today, 2000. 21(3): 141-7.
44. Masson, F., T. Calzascia, W. Di Berardino-Besson, N. de Tribolet, P. Y. Dietrich, and P. R. Walker, Brain microenvironment promotes the final functional maturation of tumor-specific effector CD8+ T cells. J Immunol, 2007. 179(2): 845-53.
45. Schlager, C., H. Korner, M. Krueger, S. Vidoli, M. Haberl, D. Mielke, et al., Effector T-cell trafficking between the leptomeninges and the cerebrospinal fluid. Nature, 2016. 530(7590): 349-53.
46. Carmi, Y., M. H. Spitzer, I. L. Linde, B. M. Burt, T. R. Prestwood, N. Perlman, et al., Allogeneic IgG combined with dendritic cell stimuli induce antitumour T-cell immunity. Nature, 2015. 521(7550): 99-104.
47. Wu, T. D., S. Madireddi, P. E. de Almeida, R. Banchereau, Y.-J. J. Chen, A. S. Chitre, et al., Peripheral T cell expansion predicts tumour infiltration and clinical response. Nature, 2020. 579(7798): 274-278.
48. Madkouri, R., C. G. Kaderbhai, A. Bertaut, C. Truntzer, J. Vincent, M. H. Aubriot-Lorton, et al., Immune classifications with cytotoxic CD8+ and Th17 infiltrates are predictors of clinical prognosis in glioblastoma. OncoImmunology, 2017: e1321186.
49. Fridman, W. H., L. Zitvogel, C. Sautes-Fridman, and G. Kroemer, The immune contexture in cancer prognosis and treatment. Nature reviews Clinical oncology, 2017. 14(12): 717.
50. Saha, D., R. L. Martuza, and S. D. Rabkin, Macrophage Polarization Contributes to Glioblastoma Eradication by Combination Immunovirotherapy and Immune Checkpoint Blockade. Cancer Cell, 2017. 32(2): 253-267 e5.
51. Deng, L., H. Liang, B. Burnette, M. Beckett, T. Darga, R. R. Weichselbaum, et al., Irradiation and anti-PD-L1 treatment synergistically promote antitumor immunity in mice. J Clin Invest, 2014. 124(2): 687-95.
52. Wu, C. T., W. C. Chen, Y. H. Chang, W. Y. Lin, and M. F. Chen, The role of PD-L1 in the radiation response and clinical outcome for bladder cancer. Sci Rep, 2016. 6: 19740.
53. Marabelle, A., H. Kohrt, C. Caux, and R. Levy, Intratumoral immunization: a new paradigm for cancer therapy. Clin Cancer Res, 2014. 20(7): 1747-56.

1. A method of increasing proliferation of CD8+ T cells comprising
exposing a target site to an alternating electric field for a period of time, the alternating electric field having a frequency and field strength,
wherein the frequency and field strength of the alternating electric field increases proliferation of CD8+ T cells at the target site.

2. A method of generating a pro-inflammatory response in a target site comprising
exposing the target site to an alternating electric field for a period of time, the alternating electric field having a frequency and field strength,
wherein the frequency and field strength of the alternating electric field generates a pro-inflammatory response at the target site.

3. The method of claim 2, wherein the pro-inflammatory response comprises one or more cytokines.

4. The method of claim 3, wherein the one or more cytokines is IFN-γ or TNF-α.

5. The method of claim 1, wherein the target site comprises stimulated CD8+ T cells.

6. The method of claim 1, wherein the target site is a site of inflammation or a site comprising a draining lymph node.

7. The method of claim 6, wherein the site of inflammation is a burn site, an infection site, or an amputation site.

8. The method of claim 7, wherein the infection site is a viral infection site.

9. The method of claim 8, wherein the viral infection site is a Pneumonia viral infection site, Meningitis viral infection site, or Herpes zoster viral infection site.

10. The method of claim 6, wherein the draining lymph node is near or adjacent to a site of inflammation.

11. The method of claim 6, wherein the site of a draining lymph node is near or adjacent to a primary tumor.

12. (canceled)

13. The method of claim 1, wherein the alternating electric field has a frequency of 150 kHz, 200 kHz, or 300 kHz.

14. The method of claim 1, wherein the period of time is 1-10 days.

15. (canceled)

16. A method of increasing proliferation of CD8+ T cells comprising
exposing CD8+ T cells to an alternating electric field for a period of time, the alternating electric field having a frequency and field strength,
wherein the frequency and field strength of the alternating electric field increases proliferation of CD8+ T cells.

17. The method of claim 16, wherein the CD8+ T cells are stimulated CD8+ T cells.

18. The method of claim 16, wherein the CD8+ T cells are in a subject.

19. The method of claim 16, wherein the CD8+ T cells are exposed to the alternating electrical field in vitro.

20. The method of claim 16, wherein the CD8+ T cells are exposed to the alternating electric field through a container suitable for cell culture.

21. The method of claim 16, further comprising a step of administering the CD8+ T cells exposed to the alternating electric field into a subject.

22. The method of claim 21, wherein the subject has an infection, cancer, a burn or has recently undergone an organ amputation.

23.-32. (canceled)
